(19) United States
(12) Patent Application Publication
(10) Pub. No.: US 2023/0147883 A1
(43) Pub. Date: May 11, 2023

(54) METHOD FOR PRODCING CARDIOMYOCYTES FROM INDUCED PLURIPOTENT STEM CELLS IN AN INTEGRATED PROCESS

(71) Applicants: Fatemeh Etezadi, Tehran (IR); Koichi Hasegawa, Kyoto (JP); Fatemeh Jahanpeyma, Tehran (IR)

(72) Inventors: Fatemeh Etezadi, Tehran (IR); Koichi Hasegawa, Kyoto (JP); Fatemeh Jahanpeyma, Tehran (IR)

(73) Assignees: Fatemeh Etezadi, Tehran (IR); Fatemeh Jahanpeyma, Tehran (IR)

(21) Appl. No.: 17/993,869

(22) Filed: Nov. 23, 2022

Related U.S. Application Data
(60) Provisional application No. 63/283,478, filed on Nov. 28, 2021.

(51) Int. Cl. C12N 5/077

(52) U.S. Cl. CPC C12N 5/0657; C12N 2506/45; C12N 2500/92; C12N 2501/415; C12N 2501/33; C12N 2533/00

(57) ABSTRACT

A method for generating a population of cardiomyocytes from induced pluripotent stem cells (iPSCs) in an integrated process. The method may comprise seeding the iPSCs on a modified surface of a modified cell culture substrate, culturing the seeded iPSCs on the modified surface of the modified cell culture substrate in an animal component-free culture medium, and differentiating the cultured iPSCs to the population of cardiomyocytes on the modified surface of the modified cell culture substrate. The modified cell culture substrate may comprise a patterned polydimethylsiloxane (PDMS) substrate, a first coating comprising a plurality of polydopamine molecules, and a second coating comprising a plurality of Laminin 511 E8 Fragment (LME8) molecules.

### CROSS-REFERENCE TO RELATED APPLICATION

This application claims the benefit of priority from U.S. Provisional Patent Application Ser. No. 63/283,478, filed on Nov. 28, 2021, entitled “SYSTEM AND METHOD FOR SINGLE-PHASE PRODUCTION OF CARDIOMYOCYTES FROM INDUCED PLURIPOTENT STEM CELLS” which is incorporated herein by reference in its entirety.

### TECHNICAL FIELD

The present disclosure is generally related to an exemplary method for producing cardiomyocytes from induced pluripotent stem cells (iPSCs) in an integrated process, and more particularly to an exemplary method for producing cardiomyocytes from iPSCs using an exemplary modified cell culture substrate.

### BACKGROUND

Induced pluripotent stem cells (iPSCs) are a type of PSCs obtained from adult somatic cells and genetically reprogrammed to exhibit similar growth and morphological properties as embryonic stem cells (ESCs). Self-renewal capacity of iPSCs and their differentiation potency towards cardiomyocyte have made them a reasonable option for producing functional cardiomyocytes that may be applicable for cardiac therapy/regeneration, cardiac safety pharmacology testing, drug discovery, etc.

Adherent mono- or multi-layer cultivation on the surface of cell culture substrates has been one of the widely-used methods for producing iPSCs-derived cardiomyocytes. However, a major challenge with mono- or multi-layer culture methods has been detachment of proliferating iPSCs and differentiating cells from the surface of cell culture substrates during the lengthy process of producing iPSCs-driven cardiomyocytes (more than 20 days). Early detachment of differentiating cells from the surface of substrates during the differentiation process of iPSCs to cardiomyocytes may prevent the completion of differentiation and maturation phase, and in turn may lead to formation of immature and unfunctional cardiomyocytes. Current methods have solved this challenge by dividing the differentiation process of iPSCs to cardiomyocytes into two or more separate phases—for example, by conducting each phase including iPSCs expansion, differentiation of iPSCs to cardiac progenitors, and differentiation of cardiac progenitors to cardiomyocytes in separate systems and processes. Performing a differentiation process in two or more phase may complicate differentiation process and may lead to spontaneous differentiation of iPSCs to unwanted cell lines, low-scale production of cardiomyocytes, and production of a low population of highly matured and functional cardiomyocyte.

Thus, there is need to develop a system and/or method capable of maintaining iPSCs, differentiating cells, and cardiomyocytes attached to the surface of a cell culture substrate during an entire process of iPSCs proliferation/expansion, differentiation, and maturation (i.e., a method that may allow for producing iPSCs-derived cardiomyocytes in an integrated process).

### SUMMARY

This summary is intended to provide an overview of the subject matter of the present disclosure, and is not intended to identify essential elements or key elements of the subject matter, nor is it intended to be used to determine the scope of the claimed implementations. Its sole purpose is to present some concepts of one or more exemplary aspects in a simplified form as a prelude to the more detailed description that is presented later. The proper scope of the present disclosure may be ascertained from the claims set forth below in view of the detailed description below and the drawings.

One or more exemplary embodiments describe an exemplary method for generating an exemplary population of cardiomyocytes from exemplary induced pluripotent stem cells (iPSCs) in an integrated process. An exemplary method may comprise seeding exemplary iPSCs on a modified surface of an exemplary modified cell culture substrate. In an exemplary embodiment, an exemplary modified cell culture substrate may comprise an exemplary patterned polydimethylsiloxane (PDMS) substrate with an exemplary stiffness of about 667 kPa. An exemplary patterned PDMS substrate may include an exemplary imprinted pattern of exemplary human fetus cardiomyocytes on a cell-contacting surface of an exemplary patterned PDMS substrate.

In an exemplary embodiment, an exemplary modified cell culture substrate may further comprise an exemplary first coating comprising a plurality of exemplary polydopamine molecules chemically attached to a cell-contacting surface of an exemplary patterned PDMS substrate. Exemplary polydopamine molecules may have a coating concentration between 47 μg/cm² and 53 μg/cm². In an exemplary embodiment, an exemplary modified cell culture substrate may further comprise an exemplary second coating covering a top surface of an exemplary first coating. An exemplary second coating may comprise a plurality of exemplary Laminin 511 E8 Fragment (LME8) molecules chemically attached to an exemplary first coating. Exemplary LME8 molecules may have a coating concentration between 0.4 μg/cm² and 0.6 μg/cm².

In one or more exemplary embodiments, an exemplary method may further comprise culturing exemplary seeded iPSCs on a modified surface of an exemplary modified cell culture substrate in an exemplary animal component-free culture medium until reaching a confluency of at least 80%. In an exemplary embodiment, an exemplary method may further comprise differentiating exemplary cultured iPSCs to an exemplary population of cardiomyocytes on a modified surface of an exemplary modified cell culture substrate.

In an exemplary embodiment, differentiating exemplary cultured iPSCs to an exemplary population of cardiomyocytes on a modified surface of an exemplary modified cell culture substrate may comprise inducing differentiation of exemplary cultured iPSCs to an exemplary population of mesoderm cells by culturing exemplary cultured iPSCs in an exemplary first growth medium comprising an exemplary Wnt activator. In an exemplary embodiment, differentiating exemplary cultured iPSCs to an exemplary population of cardiomyocytes on a modified surface of an exemplary modified cell culture substrate may further comprise inducing differentiation of an exemplary population of mesoderm cells to an exemplary population of cardiac mesoderm cells by culturing an exemplary population of mesoderm cells in an exemplary second growth medium comprising an exemplary Wnt inhibitor. In an exemplary embodiment, differentiating exemplary cultured iPSCs to an exemplary population of cardiomyocytes on a modified surface of an exemplary modified cell culture substrate may further comprise inducing differentiation of an exemplary population of cardiac mesoderm cells to an exemplary population of cardiomyocytes by culturing an exemplary population of cardiac mesoderm cells in an exemplary third growth medium comprising Insulin.

This Summary may introduce a number of concepts in a simplified format; the concepts are further disclosed within the “Detailed Description” section. This Summary is not intended to configure essential/key features of the claimed subject matter, nor is intended to limit the scope of the claimed subject matter.

### BRIEF DESCRIPTION OF THE DRAWINGS

The novel features which are believed to be characteristic of the present disclosure, as to its structure, organization, use and method of operation, together with further objectives and advantages thereof, will be better understood from the following drawings in which an exemplary embodiment will now be illustrated by way of example. It is expressly understood, however, that the drawings are for the purpose of illustration and description only and are not intended as a definition of the limits of the present disclosure. Exemplary embodiments will now be described by way of example in association with the accompanying drawings in which:

FIG. 1 illustrates an exemplary flowchart of an exemplary method for generating an exemplary population of cardiomyocytes from exemplary induced pluripotent stem cells (iPSCs) in an integrated process, consistent with one or more exemplary embodiments of the present disclosure;

FIG. 2 illustrates an exemplary flowchart of an exemplary method for imprinting an exemplary pattern of exemplary human fetus cardiomyocytes (hfCMs) on a cell-contacting surface of an exemplary PDMS substrate, consistent with one or more exemplary embodiments of the present disclosure;

FIG. 3 illustrates an exemplary method for culturing exemplary seeded iPSCs on a modified surface of an exemplary modified cell culture substrate, consistent with one or more exemplary embodiments of the present disclosure;

FIG. 4 illustrates an exemplary method for differentiating exemplary cultured iPSCs to an exemplary population of cardiomyocytes on a modified surface of an exemplary modified cell culture substrate, consistent with one or more exemplary embodiments of the present disclosure;

FIG. 5 illustrates microscopic images of hfCMs during 14 days of culture, consistent with one or more exemplary embodiments of the present disclosure;

FIG. 6 illustrates microscopic images of immunocytochemistry (ICC) analysis of exemplary isolated hfCMs, consistent with one or more exemplary embodiments of the present disclosure;

FIG. 7 illustrates graphs of atomic force microscopy (AFM)-based stiffness measurement of exemplary polydimethylsiloxane (PDMS) substrates containing an exemplary pre-polymer and an exemplary cross-linker with a weight ratio (cross-linker:pre-polymer) of 1:10 and 1:30, consistent with one or more exemplary embodiments of the present disclosure;

FIG. 8 illustrates light microscopy images of an exemplary plain PDMS substrate and an exemplary cell-imprinted PDMS substrate while culturing human iPSCs (hiPSCs), consistent with one or more exemplary embodiments of the present disclosure;

FIG. 9 illustrates scanning electron microscope (SEM) images of an exemplary plain PDMS substrate and an exemplary cell-imprinted PDMS substrate with and without culture of hiPSCs, consistent with one or more exemplary embodiments of the present disclosure;

FIG. 10 illustrates AFM images of an exemplary plain PDMS substrate and an exemplary cell-imprinted PDMS substrate visualized using AFM, consistent with one or more exemplary embodiments of the present disclosure;

FIG. 11 illustrates exemplary snapshots of an exemplary configuration of water droplets floating on a cell-contacting surface of an exemplary native/plain PDMS substrate, an exemplary plasma (PL)-treated PDMS substrate, and an exemplary polydopamine (PD)-coated PDMS substrate, consistent with one or more exemplary embodiments of the present disclosure;

FIG. 12 illustrates charts of contact angle analysis of water droplets with respect to the cell-contacting surface of an exemplary plain/native PDMS substrate, an exemplary PL-treated PDMS substrate, and an exemplary PD-coated PDMS substrate immediately after forming water droplets (day 0) and after 7 days, consistent with one or more exemplary embodiments of the present disclosure;

FIG. 13 illustrates light microscopy images of exemplary cultured hiPSCs on the cell-contacting surface of exemplary untreated and biochemically-modified PDMS substrates, consistent with one or more exemplary embodiments of the present disclosure;

FIG. 14 illustrates charts of proliferation and survival analysis of hiPSCs—based on total cell number and number of viable hiPSCs—after 5 days of culture on exemplary biochemically-modified PDMS substrates coated with exemplary extracellular matrix (ECM) proteins, consistent with one or more exemplary embodiments of the present disclosure;

FIG. 15 illustrates graphs of flow cytometry analysis of exemplary NANOG-positive hiPSCs on fourth day of culture to assess pluripotency maintenance of exemplary hiPSCs on the modified surface of an exemplary PL-treated PDMS substrate with VTN-N coating, an exemplary PD-coated PDMS substrate with Vitronectin (VTN-N) coating, an exemplary PD-coated PDMS substrate with Laminin-511 E8 fragment (LME8) coating, and an exemplary tissue culture polystyrene (TCPS) as control, consistent with one or more exemplary embodiments of the present disclosure;

FIG. 16 illustrates timeline chart of an exemplary procedure of cardiomyocyte differentiation and maturation, consistent with one or more exemplary embodiments of the present disclosure;

FIG. 17 illustrates light microscopy images of exemplary hiPSCs after mesoderm induction on the modified surface of an exemplary PL-treated PDMS substrate with VTN-N coating, an exemplary PD-coated PDMS substrate with VTN-N coating, and an exemplary PD-coated PDMS substrate with LME8 coating, consistent with one or more exemplary embodiments of the present disclosure;

FIG. 18 illustrates light microscopy images of exemplary hiPSCs during their differentiation to cardiomyocytes (on day (1), day (5), day (14), and day (21) of differentiation) on the modified surface of an exemplary PD-coated PDMS substrate with LME8 coating and an exemplary LME8-coated TCPS (as an exemplary control), consistent with one or more exemplary embodiments of the present disclosure;

FIG. 19 illustrates images of surface characterization of an exemplary PD-coated PDMS substrate with LME8 coating compared to an exemplary PDMS substrate without biochemical and ECM protein modifications by light microscopy, SEM, and AFM, consistent with one or more exemplary embodiments of the present disclosure;

FIG. 20 illustrates bar graph of root-mean-square (RMS) roughness analysis of an exemplary PD-coated PDMS substrate with LME8 coating without exemplary hiPSCs, an exemplary PDMS substrate without biochemical and ECM protein modification, and an exemplary PD-coated PDMS substrate with LME8 coating during expansion of exemplary hiPSCs, consistent with one or more exemplary embodiments of the present disclosure;

FIG. 21 illustrates charts of expression level analysis of exemplary differentiation genes during differentiation of exemplary hiPSCs to cardiomyocytes on the modified surface of an exemplary PD-coated PDMS substrate with LME8 coating, compared to the expression level of exemplary differentiation genes during differentiation of exemplary hiPSCs to cardiomyocytes on the modified surface of an exemplary LME8-coated TCPS (as an exemplary control), consistent with one or more exemplary embodiments of the present disclosure;

FIG. 22 illustrates microscopic images of immunostaining assay of exemplary cardiac markers, myosin heavy chain 7 gene (MYH7), and cardiac troponin T protein (cTnT) on day (50) of cardiac differentiation, consistent with one or more exemplary embodiments of the present disclosure;

FIG. 23 illustrates graphs of flow cytometry analysis of hiPSCs-derived cardiomyocytes to evaluate cTnT expression, consistent with one or more exemplary embodiments of the present disclosure;

FIG. 24 illustrates light microscopy images captured from the modified surface of an exemplary cell-imprinted PDMS substrate with 667 kPa (1:10) and 106 kPa (1:30) stiffness and the surface of an exemplary plain PDMS substrate (i.e., without topographical modification) with 667 kPa (1:10) and 106 kPa (1:30) stiffness (all of them coated with polydopamine and LME8) during differentiation of exemplary hiPSCs to cardiomyocytes (on days (1), (8), and (21)), consistent with one or more exemplary embodiments of the present disclosure; and

FIG. 25 illustrates charts of expression level analysis of exemplary differentiation genes during differentiation of exemplary hiPSCs to cardiomyocytes over an exemplary cell-imprinted PDMS substrate with 667 kPa (1:10) and 106 kPa (1:30) stiffness and an exemplary plain PDMS substrate with 667 kPa (1:10) and 106 kPa (1:30) stiffness (all of them coated with polydopamine and LME8), consistent with one or more exemplary embodiments of the present disclosure.

### DETAILED DESCRIPTION

In the following detailed description, numerous specific details are set forth by way of examples to provide a thorough understanding of the relevant teachings related to the exemplary embodiments. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in one or more exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be plain to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Disclosed herein is an exemplary method for generating an exemplary population of cardiomyocytes from exemplary induced pluripotent stem cells (iPSCs) in an integrated process. In one or more exemplary embodiments, an exemplary method may take advantage of an exemplary modified cell culture substrate to generate iPSCs-derived cardiomyocytes in a large scale (e.g., more than about 10⁵ cardiomyocytes/cm²) and in an integrated process. “Integrated process” may refer to an exemplary process that may combine two or more phases of producing iPSCs-derived cardiomyocytes—e.g., an exemplary phase/process of iPSCs proliferation/expansion and an exemplary phase/process of iPSCs differentiation to cardiomyocytes—into a single cell culture substrate. “iPSCs” may refer to exemplary adult cells that have been created to resemble embryonic stem cells (ESCs). iPSCs may be induced in vitro to express certain factors and genes such as Oct3/4, Sox2, Klf4, and c-Myc that are used in ESCs. iPSCs may have regenerative properties and have been shown to be similar in characteristics and abilities to ESCs. Using an exemplary modified cell culture substrate, as disclosed in one or more exemplary embodiments, may prevent detachment of differentiating cells from a cell-contacting surface of an exemplary substrate and may lead to production of a significantly high population of cardiomyocytes (more than about 10⁵ cardiomyocytes/cm²).

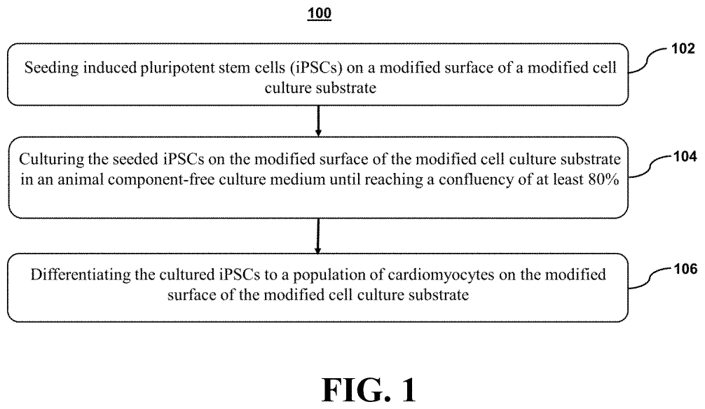

FIG. 1 illustrates an exemplary flowchart of exemplary method 100 for generating an exemplary population of cardiomyocytes from exemplary iPSCs in an integrated process, consistent with one or more exemplary embodiments of the present disclosure. In one or more exemplary embodiments, exemplary method 100 may comprise: seeding exemplary iPSCs on a modified surface of an exemplary modified cell culture substrate (step 102); culturing exemplary seeded iPSCs on a modified surface of an exemplary modified cell culture substrate in an exemplary animal component-free culture medium until reaching a confluency of at least 80% (step 104); and differentiating exemplary cultured iPSCs to exemplary population of cardiomyocytes on a modified surface of an exemplary modified cell culture substrate (step 106).

In further detail with respect to step 102, step 102 may include seeding exemplary iPSCs on a modified surface of an exemplary modified cell culture substrate. In an exemplary embodiment, seeding exemplary iPSCs on a modified surface of an exemplary modified cell culture substrate may comprise seeding at least 1×10³ iPSCs (e.g., 1×10⁵ iPSCs) on a modified surface of an exemplary modified cell culture substrate. “Cell seeding” may refer to uniformly spreading cells to a culture vessel to form a monolayer. In an exemplary embodiment, seeding at least 1×10³ iPSCs on a modified surface of an exemplary modified cell culture substrate may comprise preparing an exemplary cell suspension (a homogenous solution) by mixing at least 1×10³ human iPSCs (hiPSCs) in a certain amount (e.g., 15 mL) of an exemplary animal component-free culture medium and spreading an exemplary cell suspension on a modified surface of an exemplary modified cell culture substrate (using a sampler). In one or more exemplary embodiments, an exemplary animal component-free culture medium may comprise a Dulbecco's Modified Eagle Medium (DMEM) F-12 comprising at least one of L-ascorbic acid-2-phosphate magnesium, sodium selenium, fibroblast growth factor 2 (FGF2), Insulin, NaHCO₃, transferrin, and transforming growth factor beta 1 (TGFβ1).

In one or more exemplary embodiments, an exemplary modified cell culture substrate set forth in step 102 may comprise an exemplary patterned polydimethylsiloxane (PDMS) substrate. “Polydimethylsiloxane (PDMS)” may refer to a silicone elastomer that may be used in lab-on-a-chip or microfluidic applications to form exemplary devices with defined microstructures. Fabrication of an exemplary PDMS may include mixing an exemplary ratio of an exemplary elastomer/pre-polymer with an exemplary curing agent, degassing, and baking for one or more hours at an exemplary temperature level such as 90° C. In an exemplary embodiment, an exemplary modified cell culture substrate may comprise an exemplary patterned PDMS substrate with a stiffness of about 667 kPa. In an exemplary embodiment, an exemplary modified cell culture substrate may comprise an exemplary patterned PDMS substrate with a stiffness of about 667 kPa. “Stiffness” may also be known as Young's modulus, elastic modulus, tensile modulus, and/or modulus of elasticity. SI unit (i.e., the international system of units) for Young's modulus is Pascal (Pa). Ratio of an exemplary elastomer/pre-polymer to an exemplary curing agent (cross-linker) may determine stiffness (Young's modulus) of a PDMS substrate. In an exemplary embodiment, an exemplary patterned PDMS substrate with a stiffness of about 667 kPa may comprise an exemplary elastomer/pre-polymer solution and an exemplary curing agent solution (cross-linker) with a weight ratio (curing agent:pre-polymer) of about 1:30. In one or more exemplary embodiments, an exemplary pre-polymer solution/component may comprise at least one of dimethyl siloxane, dimethyl vinyl terminated, dimethylvinylated and trimethylated silica, tetra (trimethoxysiloxy) silane, ethyl benzene, and a combination thereof. In one or more exemplary embodiments, an exemplary curing agent/cross-linker solution/component may comprise at least one of dimethyl methylhydrogen siloxane, dimethyl siloxane, dimethylvinylated silica, trimethylated silica, tetramethyl tetravinyl cyclotetra siloxane, ethyl benzene, and a combination thereof.

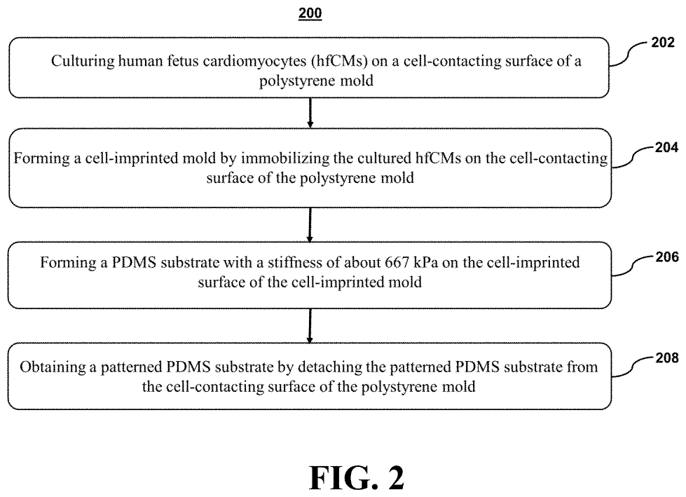

Furthermore, in an exemplary embodiment, an exemplary patterned PDMS substrate with a stiffness of about 667 kPa may comprise an exemplary imprinted pattern of exemplary human fetus cardiomyocytes (hfCMs) on a cell-contacting surface (i.e., a surface to be in contact with cells) of an exemplary patterned PDMS substrate. An exemplary imprinted pattern of exemplary hfCMs may refer to an exemplary 3-dimensional (3D) topographical structure of exemplary hfCMs being imprinted on a cell-contacting surface of an exemplary PDMS substrate. In an exemplary embodiment, details of step 102 for preparing an exemplary patterned PDMS substrate are described in context of elements presented in FIG. 2.

FIG. 2 illustrates an exemplary flowchart of exemplary method 200 for imprinting an exemplary pattern of exemplary hfCMs on a cell-contacting surface of an exemplary PDMS substrate, consistent with one or more exemplary embodiments of the present disclosure. In an exemplary implementation, exemplary method 200 may include culturing exemplary hfCMs on a cell-contacting surface of an exemplary polystyrene mold (step 202), forming an exemplary cell-imprinted mold by immobilizing exemplary cultured hfCMs on a cell-contacting surface of an exemplary polystyrene mold (step 204), forming an exemplary PDMS substrate with a stiffness of about 667 kPa on a cell-imprinted surface of an exemplary cell-imprinted mold (step 206), and obtaining an exemplary patterned PDMS substrate by detaching an exemplary patterned PDMS substrate from a cell-contacting surface of an exemplary polystyrene mold (step 208).

In further detail with respect to step 202, step 202 may include culturing exemplary hfCMs on a cell-contacting surface of an exemplary polystyrene mold. In an exemplary embodiment, culturing exemplary hfCMs on a cell-contacting surface of an exemplary polystyrene mold may include culturing exemplary hfCMs on a cell-contacting surface of an exemplary polystyrene mold in DMEM/Ham's F12 at a temperature level of about 37° C. and a CO₂ atmosphere with about 5% concentration (for about 10-16 days). In an exemplary implementation, an exemplary DMEM/Ham's F12 medium may comprise fetal bovine serum (e.g., 10% FBS), penicillin with a concentration between about 95 U/mL and 105 U/mL, and streptomycin with a concentration between about 95 μg/mL and 105 μg/mL. In an exemplary implementation, exemplary hfCMs may be cultured on a cell-contacting surface of an exemplary polystyrene mold until reaching a confluency between about 70% and 100%.

In further detail with respect to step 204, step 204 may include forming an exemplary cell-imprinted mold by immobilizing exemplary cultured hfCMs on a cell-contacting surface of an exemplary polystyrene mold. In an exemplary embodiment, immobilizing exemplary cultured hfCMs on a cell-contacting surface of an exemplary polystyrene mold may include adding an exemplary glutaraldehyde solution with a concentration between about 3% (w/v) and 5% (w/v) to exemplary cultured hfCMs on a cell-contacting surface of an exemplary polystyrene mold. In an exemplary implementation, a cell-contacting surface of an exemplary polystyrene mold may be treated with an exemplary glutaraldehyde solution for a time duration between 40 and 50 minutes followed by washing with an exemplary deionized water.

In further detail with respect to step 206, step 206 may include forming an exemplary PDMS substrate with a stiffness of about 667 kPa on a cell-imprinted surface of an exemplary cell-imprinted mold. In an exemplary implementation, forming an exemplary PDMS substrate with a stiffness of about 667 kPa on a cell-imprinted surface of an exemplary cell-imprinted mold may include pouring (drop-wisely) an exemplary mixture of an exemplary pre-polymer and an exemplary curing agent with a weight ratio (curing agent:pre-polymer) of about 1:30 on a cell-imprinted surface of an exemplary cell-imprinted mold followed by placing an exemplary cell-imprinted mold at a temperature level between about 34° C. and 39° C. for about 8-24 hours.

In further detail with respect to step 208, step 208 may include obtaining an exemplary patterned PDMS substrate by detaching an exemplary patterned PDMS substrate from a cell-contacting surface of an exemplary polystyrene mold. In one or more exemplary implementations, an exemplary patterned PDMS substrate may be first swelled on a cell-contacting surface of an exemplary polystyrene mold using an exemplary nonpolar organic solvent such as toluene, dichloromethane, hydrocarbons, hexane, and/or exemplary organic solvents. An exemplary patterned PDMS substrate may then be peeled off from an exemplary polystyrene mold. An exemplary detached patterned PDMS substrate may be washed with a 0.5-1.5 M NaOH solution on a shaker and sterilized under ultraviolet (UV) light.

With further reference to step 102 of exemplary method 100 (FIG. 1), an exemplary modified cell culture substrate (i.e., an exemplary patterned PDMS substrate with a stiffness of about 667 kPa) may further comprise an exemplary first coating. In an exemplary embodiment, an exemplary first coating may comprise a plurality of exemplary polydopamine molecules chemically attached to a cell-contacting surface of an exemplary patterned PDMS substrate. In an exemplary embodiment, exemplary polydopamine molecules may have a coating concentration between about 47 μg/cm² and 53 μg/cm². In an exemplary embodiment, exemplary polydopamine molecules may be chemically attached to a cell-contacting surface of an exemplary patterned PDMS substrate through an exemplary reaction between exemplary functional groups of exemplary PDMS polymer chains and catechol and amine groups of exemplary polydopamine molecules. In an exemplary implementation, an exemplary first coating may be formed on a cell-contacting surface of an exemplary patterned PDMS substrate by immersing a cell-contacting surface of an exemplary patterned PDMS substrate (with a stiffness of about 667 kPa) in an exemplary dopamine solution (about 0.007-0.02% w/v in Tris-HCl, pH 8-9) for a time duration between about 8 and 24 hours followed by washing with deionized water and sterilization under UV light.

In an exemplary embodiment, an exemplary modified cell culture substrate (i.e., an exemplary patterned PDMS substrate with a stiffness of about 667 kPa) may further comprise an exemplary second coating covering a top surface of an exemplary first coating. In an exemplary embodiment, an exemplary second coating may comprise a plurality of exemplary Laminin 511 E8 Fragment (LME8) molecules chemically attached to an exemplary first coating. In an exemplary embodiment, exemplary LME8 molecules may have a coating concentration between about 0.4 μg/cm² and 0.6 μg/cm². In an exemplary embodiment, exemplary LME8 molecules may be chemically attached to an exemplary first coating through an exemplary reaction between catechol and amine groups of exemplary polydopamine molecules and exemplary functional groups of exemplary LME8 molecules including, but not limited to, amine and carboxyl groups. “Laminin 511 E8” (150 kDa) is a truncated form of Laminin 511 that may be one of exemplary extracellular matrix (ECM) proteins. Exemplary isoforms of Laminin E8 may serve as functionally minimal forms of Laminin having a full capability for coupling to integrins. Laminin 511 E8 may comprise a C-terminal end of exemplary beta 1, alpha 5, and gamma 1 chains.

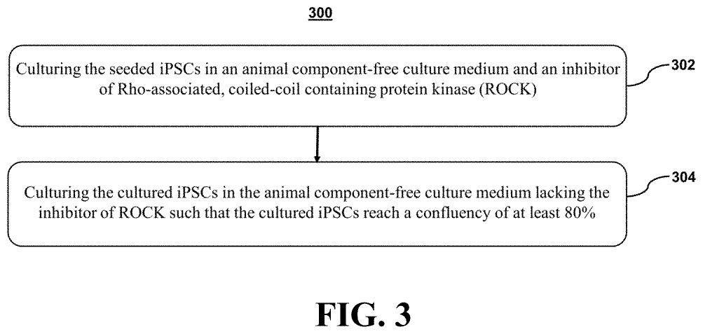

In further detail with respect to step 104, step 104 of exemplary method 100 may include culturing exemplary seeded iPSCs on a modified surface of an exemplary modified cell culture substrate in an exemplary animal component-free culture medium until reaching a confluency of at least 80%. “Confluency” may refer to the percentage of a surface of a culture substrate that is covered by adherent cells. In an exemplary implementation, culturing exemplary seeded iPSCs on a modified surface of an exemplary modified cell culture substrate in an exemplary animal component-free culture medium may include culturing exemplary seeded iPSCs on a modified surface of an exemplary modified cell culture substrate in an exemplary animal component-free culture medium until reaching a confluency of between about 90% and 100%. “Animal component-free culture medium” may refer to a culture medium that does not contain any materials that are derived directly from animal body fluid or tissue. In an exemplary embodiment, details of step 104 for culturing exemplary seeded iPSCs on a modified surface of an exemplary modified cell culture substrate are described in context of elements presented in FIG. 3. FIG. 3 illustrates exemplary method 300 for culturing exemplary seeded iPSCs on a modified surface of an exemplary modified cell culture substrate, consistent with one or more exemplary embodiments of the present disclosure. In an exemplary embodiment, method 300 may include: culturing exemplary seeded iPSCs in an exemplary animal component-free culture medium and an exemplary inhibitor of Rho-associated, coiled-coil containing protein kinase (ROCK) (step 302), and culturing exemplary cultured iPSCs in an exemplary animal component-free culture medium lacking an exemplary inhibitor of ROCK such that exemplary cultured iPSCs reach a confluency of at least 80% (step 304).

In further detail with regards to step 302, step 302 may include culturing exemplary seeded iPSCs in an exemplary animal component-free culture medium and an exemplary inhibitor of ROCK. In an exemplary implementation, culturing exemplary seeded iPSCs may comprise culturing exemplary seeded iPSCs in an exemplary animal component-free culture medium and an exemplary inhibitor of ROCK for a time duration of at least 24 hours (at about 37° C. and about 5% CO₂). In one or more exemplary embodiments, an exemplary animal component-free culture medium may comprise DMEM F-12 comprising at least one of L-ascorbic acid-2-phosphate magnesium, sodium selenium, fibroblast growth factor 2 (FGF2), Insulin, NaHCO₃, transferrin, and transforming growth factor beta 1 (TGFβ1). In one or more exemplary embodiments, an exemplary inhibitor of ROCK may include, but is not limited to, at least one of Y27632, fasudil, and Azaindole 1. In an exemplary implementation, an exemplary animal component-free culture medium may comprise Y27632 with a concentration between about 8 μM and 12 μM.

In an exemplary embodiment, step 304 may include culturing exemplary cultured iPSCs in an exemplary animal component-free culture medium lacking an exemplary inhibitor of ROCK such that exemplary cultured iPSCs reach a confluency of at least 80%. In an exemplary embodiment, culturing exemplary cultured iPSCs in an exemplary animal component-free culture medium lacking an exemplary inhibitor of ROCK may include culturing exemplary cultured iPSCs in an exemplary animal component-free culture medium lacking an exemplary inhibitor of ROCK for a time duration between 24 and 72 hours (at about 37° C. and about 5% CO₂) such that exemplary cultured iPSCs reach a confluency between about 80% and 100%.

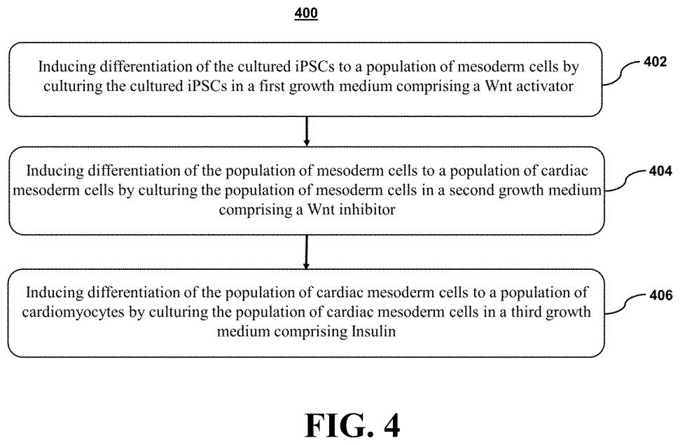

In further detail with respect to step 106, step 106 may include differentiating exemplary cultured iPSCs to an exemplary population of cardiomyocytes on a modified surface of an exemplary modified cell culture substrate. In an exemplary embodiment, details of step 106 for differentiating exemplary cultured iPSCs to an exemplary population of cardiomyocytes on a modified surface of an exemplary modified cell culture substrate are described in context of elements presented in FIG. 4. FIG. 4 illustrates exemplary method 400 for differentiating exemplary cultured iPSCs to an exemplary population of cardiomyocytes on a modified surface of an exemplary modified cell culture substrate, consistent with one or more exemplary embodiments of the present disclosure. In an exemplary embodiment, exemplary method 400 may include: inducing differentiation of exemplary cultured iPSCs to an exemplary population of mesoderm cells by culturing exemplary cultured iPSCs in an exemplary first growth medium comprising an exemplary Wnt activator (step 402); inducing differentiation of an exemplary population of mesoderm cells to an exemplary population of cardiac mesoderm cells by culturing an exemplary population of mesoderm cells in an exemplary second growth medium comprising an exemplary Wnt inhibitor (step 404); and inducing differentiation of an exemplary population of cardiac mesoderm cells to an exemplary population of cardiomyocytes by culturing an exemplary population of cardiac mesoderm cells in an exemplary third growth medium comprising Insulin (step 406).

In further detail with respect to step 402, step 402 may include inducing differentiation of exemplary cultured iPSCs to an exemplary population of mesoderm cells by culturing exemplary cultured iPSCs in an exemplary first growth medium comprising an exemplary Wnt activator. In an exemplary embodiment, inducing differentiation of exemplary cultured iPSCs to an exemplary population of mesoderm cells by culturing exemplary cultured iPSCs in an exemplary first growth medium comprising an exemplary Wnt activator may comprise inducing differentiation of exemplary cultured iPSCs to an exemplary population of mesoderm cells by culturing exemplary cultured iPSCs in Roswell Park Memorial Institute Medium (RPMI) comprising CHIR99021 with a concentration between 10 μM and 14 μM for a time duration between 18 and 30 hours. An exemplary Wnt activator may include an exemplary small molecule and/or growth factor capable of activating an exemplary signaling pathway of (TGF)-β (transforming growth factor β) in exemplary cultured iPSCs. In one or more exemplary embodiments, exemplary growth factors for mesodermal induction may include, but are not limited to, bone morphogenetic protein (BMP)4, Activin A, and other exemplary growth factors that may be useful for mesodermal induction. In one or more exemplary embodiments, exemplary small molecules for mesodermal induction may include, but are not limited to, GSK-30 inhibitors (CHIR99021). Exemplary inhibitors of (TGF)-β signaling pathway may raise endogenous levels of BMP2/4 and may lead to indirect activation of TGF-β signaling pathway.

In an exemplary embodiment, step 404 may include inducing differentiation of an exemplary population of mesoderm cells to an exemplary population of cardiac mesoderm/progenitor cells by culturing an exemplary population of mesoderm cells in an exemplary second growth medium comprising an exemplary Wnt inhibitor. In an exemplary implementation, inducing differentiation of an exemplary population of mesoderm cells to an exemplary population of cardiac mesoderm cells by culturing an exemplary population of mesoderm cells in an exemplary second growth medium comprising an exemplary Wnt inhibitor may include inducing differentiation of an exemplary population of mesoderm cells to an exemplary population of cardiac mesoderm cells by culturing an exemplary population of mesoderm cells in RPMI comprising XAV939 with a concentration between 1.5 μM and 2.5 μM for a time duration between 48 and 100 hours. Induction of cardiac mesoderm/progenitors may be accomplished by removing TGF-β signaling pathway activators followed by adding growth factors including, but not limited to, vascular endothelial growth factor and/or fibroblast growth factor-2 that may induce ERK signaling pathway. Cardiac mesoderm induction may be accomplished by adding one or more exemplary small molecules including, but not limited to, XAV939, IWR-1, IWP-2, and KY02111 that may inhibit Wnt signaling pathway. Exemplary Wnt inhibitors may drive mesodermal lineage towards cardiac progenitor lineage and may inhibit development of endothelial cell lineage and smooth muscle.

In further detail with respect to step 406, step 406 may include inducing differentiation of an exemplary population of cardiac mesoderm cells to an exemplary population of cardiomyocytes by culturing an exemplary population of cardiac mesoderm cells in an exemplary third growth medium comprising Insulin. In an exemplary implementation, inducing differentiation of an exemplary population of cardiac mesoderm cells to an exemplary population of cardiomyocytes by culturing an exemplary population of cardiac mesoderm cells in an exemplary third growth medium comprising Insulin may include inducing differentiation of an exemplary population of cardiac mesoderm cells in RPMI comprising Insulin (0.5-1.5 μg) for a time duration between 5 and 15 days.

An exemplary method 100 may prevent outgrowth of fibroblasts and may lead to production of a significantly high population of cardiomyocytes (e.g., 10⁵ cells) with mature structural, metabolic, ventricular, electrophysiological, and contractile phenotype. An exemplary modified cell culture substrate may be capable of maintaining exemplary cultured iPSCs and exemplary differentiating cells attached/adherent to a modified surface of an exemplary modified cell culture substrate for 25 days or more. An exemplary method 100 for producing cardiomyocytes may increase an exemplary population of mature structural, metabolic, electrophysiological, ventricular, and contractile phenotype.

### EXAMPLES

Hereinafter, one or more exemplary embodiments will be described in further detail with reference to examples. It will be obvious to a person having ordinary skill in the art that these examples may be for illustrative purposes only and are not to be interpreted to limit the scope of the present disclosure.

### Example 1: Isolation of Human Fetus Cardiomyocytes (hfCMs)

In this example, cardiomyocytes (hfCMs) were isolated from human fetus samples. Exemplary heart tissues were harvested with an incision and chopped to small pieces (˜1 cm) from human fetus. The extracted heart tissues were washed three times in phosphate-buffered saline (PBS) buffer containing 3× penicillin (100 IU/mL)-streptomycin (100 μg/mL). Subsequently, exemplary washed heart tissues were digested, in an incubator, with 0.02 mg/mL collagenase type II for 1 hour. Exemplary digested heart tissues were centrifuged at 300×g for 5 minutes and the centrifugation supernatant was discarded. The cell pellets obtained from centrifugation were resuspended in Dulbecco's Modified Eagle's Medium (DMEM)/Ham's F12 comprising 10% fetal bovine serum (FBS), 100 U/mL penicillin and 100 μg/mL streptomycin. Cell pellets were then embedded in cell culture plates (precoated with collagen) and retained in a humidified incubator (37° C., 5% CO₂). The isolated hfCMs were cultured for about 14 days to become mature and were visually inspected under microscope on day 3, day 7, and day 14 of cell culture process until reaching a confluent monolayer demonstrating normal structure of CMs.

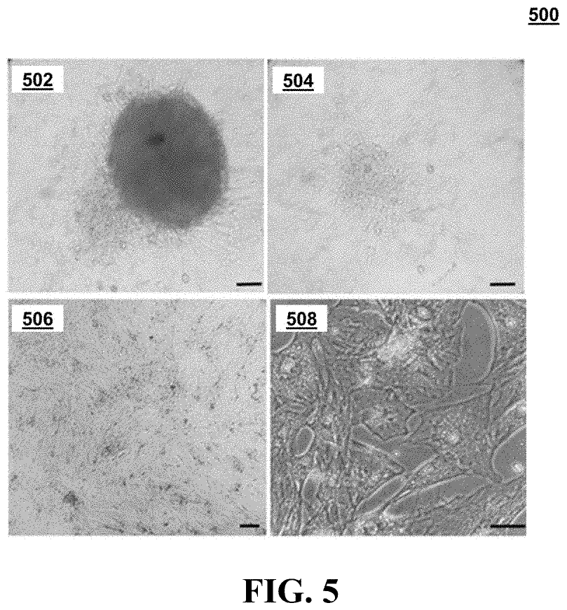

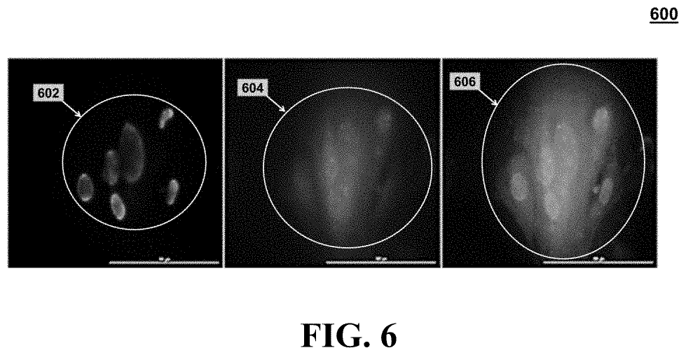

FIG. 5 illustrates microscopic images 500 of hfCMs during 14 days of culture, consistent with one or more exemplary embodiments of the present disclosure. In further detail with respect to microscopic images 500, microscopic image 502 shows day 3 of hfCMs culture, microscopic image 504 shows day 7 of hfCMs culture, microscopic image 506 shows day 14 of hfCMs culture, and microscopic image 508 shows a confluent monolayer of hfCMs on an exemplary tissue culture polystyrene (TCPS) plate. Meanwhile, exemplary isolated hfCMs were characterized by immunocytochemistry (ICC) staining of an exemplary cardiac marker protein (i.e., α-actinin) expressed on the surface of exemplary isolated hfCMs. FIG. 6 illustrates microscopic images 600 of ICC analysis of exemplary isolated hfCMs, consistent with one or more exemplary embodiments of the present disclosure. As shown in FIG. 6, stained region 602, stained region 604, and stained region 606 in microscopic images 600 demonstrate the expression of α-actinin markers on the surface of exemplary isolated hfCMs.

### Example 2: Culturing Human Induced Pluripotent Stem Cells (hiPSCs)

In this example, exemplary hiPSCs (253G1 cell line) were cultured. Briefly, exemplary hiPSCs were cultured using an animal component-free culture medium for human embryonic stem cells (e.g., TeSR-E8™), and were incubated at about 37° C., 5% CO₂, and 95% humidified atmosphere. Exemplary hiPSCs were passaged/sub-cultured until reaching a 70% confluency. “Passaging” may refer to a technique that may provide for harvesting cells from a culture and transferring them to one or more vessels containing a fresh growth medium to keep cells alive and growing for extended periods of time. To evaluate cellular viability and proliferation, exemplary hiPSCs were analyzed using an automated cell counter. In addition, the distribution and adhesion of exemplary hiPSCs were visually inspected using an inverted microscope equipped with a camera.

### Example 3: Preparation of Polydimethylsiloxane (PDMS) Substrates with Different Stiffness Values

In this example, a plurality of PDMS substrates with different stiffness values were fabricated. PDMS may comprise a pre-polymer and a curing agent; the ratio of pre-polymer to cross-linker may determine the stiffness value of a PDMS substrate. “Stiffness” may also be referred to as Young's modulus, elastic modulus, tensile modulus, and/or modulus of elasticity. The SI unit (i.e., the international system of units) for Young's modulus is Pascal (Pa). In this example, a plurality of PDMS substrates (without surface modification) having different stiffness values ranging from about 106 kPa to about 667 kPa were prepared to identify an optimal stiffness value for culturing and differentiating hiPSCs to cardiomyocytes.

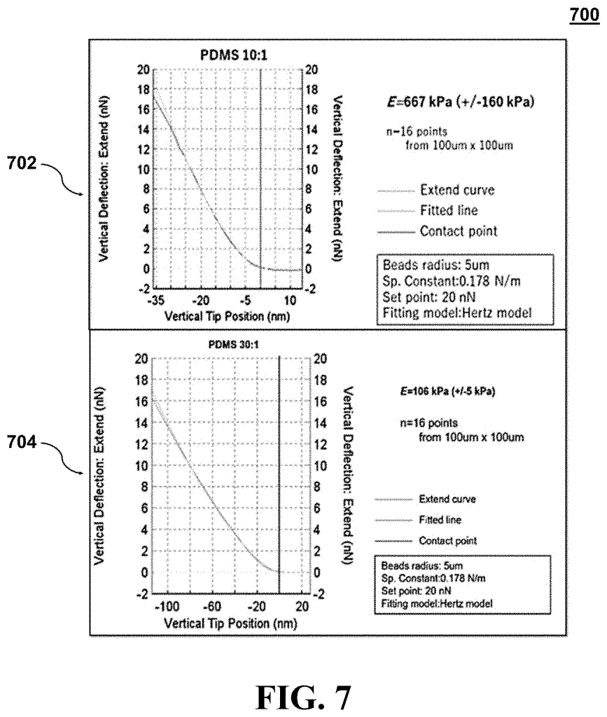

In this example, an exemplary PDMS substrate was synthesized using an exemplary commercial kit containing an exemplary base/pre-polymer component and an exemplary curing agent/cross-linker component. In an exemplary implementation, an exemplary PDMS pre-polymer (i.e., silicone resin) and an exemplary cross-linker were mixed in two weight ratios (curing agent:pre-polymer), 1:10 and 1:30. For example, to prepare an exemplary 1:10 mixture, 10 g of silicon resin was added to 1 g curing agent. The prepared exemplary 1:10 and 1:30 mixtures were stirred and degassed for 60 minutes using a vacuum oven. Then, exemplary mixtures were poured, separately, onto an exemplary non-topographical polystyrene substrate and placed in an oven at about 37° C. for about 18-24 hours. Subsequently, exemplary PDMS substrates fabricated using exemplary 1:10 and 1:30 mixtures were peeled off exemplary polystyrene substrates. Stiffness of exemplary PDMS substrates containing 1:10 and 1:30 ratios of pre-polymer and cross-linker was evaluated using atomic force microscopy (AFM). FIG. 7 illustrates graphs 700 of AFM-based stiffness measurement of exemplary PDMS substrates containing an exemplary pre-polymer and an exemplary cross-linker with a weight ratio (cross-linker:pre-polymer) of 1:10 and 1:30, consistent with one or more exemplary embodiments of the present disclosure. AFM-based stiffness measurement of exemplary PDMS substrates demonstrated that an exemplary PDMS substrate containing an exemplary pre-polymer and an exemplary cross-linker with a weight ratio (cross-linker:pre-polymer) of 1:10 may have a stiffness value of about 667 kPa (Graph 702), and an exemplary PDMS substrate containing an exemplary cross-linker with a weight ratio (cross-linker:pre-polymer) of 1:30 may have a stiffness value of about 106 kPa (Graph 704). Both of exemplary 1:10 and 1:30 PDMS mixtures may have a stiffness value lower than TCPS that may have a stiffness value of about 3 GPa.

### Example 4: Imprinting the Pattern of hfCMs on the Surface of PDMS Substrate

In this example, exemplary hfCMs—isolated based on an exemplary method set forth in “Example 1”—were cultured until reaching 100% confluency and fixed/immobilized on a mold (e.g., a polystyrene substrate) using 4% glutaraldehyde solution, for 45 minutes, and were further washed with deionized water to produce an exemplary cell-imprinted mold. Subsequently, an exemplary pre-polymer (silicone resin) and an exemplary cross-linker were mixed with two different weight ratios (curing agent:pre-polymer), 1:10 and 1:30, and degassed. The prepared exemplary mixtures were separately poured onto an exemplary cell-imprinted mold and placed at about 37° C. for 8-24 hours. Then, exemplary fabricated (i.e., cured) PDMS substrates (with a stiffness of about 106 kPa and 667 kPa) were carefully detached from an exemplary cell-imprinted mold (polystyrene substrate). The detached exemplary PDMS substrates were washed with 1 M NaOH on a shaker—to remove cell debris or unwanted components that may have been remained on the surface of exemplary detached PDMS substrates—and were further sterilized under ultra violet (UV) light. In an exemplary implementation, exemplary detached PDMS substrates may have an exemplary topography of hfCMs on their upper surface. Table 1 below sets forth exemplary PDMS substrates fabricated in one or more exemplary embodiments and their exemplary mechanical properties (i.e., stiffness). The prepared exemplary PDMS substrates were characterized by light microscopy, scanning electron microscope (SEM), and AFM—with and/or without hiPSCs culture on the surface of exemplary PDMS substrates.

TABLE 1

| | Stiffness (Pa) | |
|---|---|---|
| Substrates | (ratio of curing agent:pre-polymer) | |
| Plain/Native PDMS | 1:10 (~667 kPa) | 1:30 (~106 kPa) |
| Cell-Imprinted PDMS | 1:10 (~667 kPa) | 1:30 (~106 kPa) |

| | |
|---|---|
| Tissue Culture Polystyrene (TCPS) | Control (3 GPa) |

List of fabricated PDMS substrates and their mechanical properties, consistent with exemplary embodiments of the present disclosure.

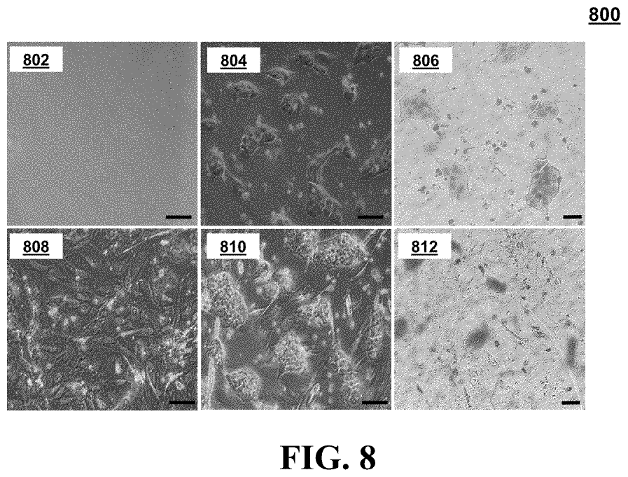

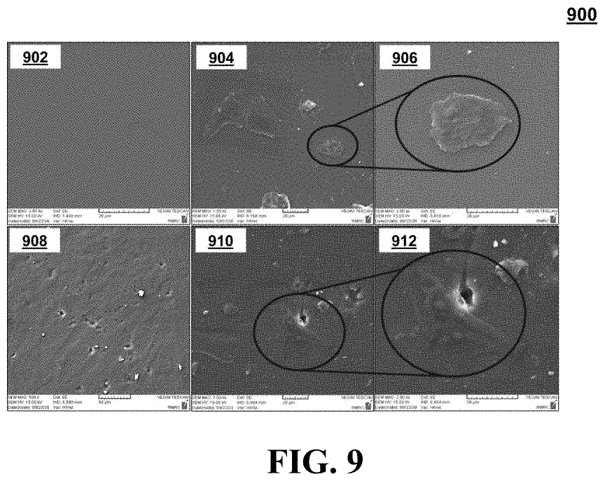

FIG. 8 illustrates light microscopy images 800 of an exemplary plain PDMS substrate and an exemplary cell-imprinted PDMS substrate while culturing hiPSCs, consistent with one or more exemplary embodiments of the present disclosure. In further detail with respect to light microscopy images 800, microscopic image 802 shows an exemplary plain PDMS substrate and microscopic image 808 shows an exemplary cell-imprinted PDMS substrate at ×20 magnifications (scale bars=200 μm) while culturing hiPSCs. Microscopic image 804 and microscopic image 810 illustrate, respectively, an exemplary plain PDMS substrate and an exemplary cell-imprinted PDMS substrates at ×40 magnifications (scale bars=200 μm) while culturing hiPSCs. Microscopic image 806 show crystal violet staining of hiPSCs cultured on an exemplary plain PDMS substrate while culturing hiPSCs and microscopic image 812 shows an exemplary cell-imprinted PDMS substrate (at ×20 magnifications (scale bars=200 μm)) while culturing hiPSCs. FIG. 9 illustrates SEM images 900 of an exemplary plain PDMS substrate and an exemplary cell-imprinted PDMS substrate with and without culture of hiPSCs, consistent with one or more exemplary embodiments of the present disclosure. Referring to SEM images 900, microscopic image 902 and microscopic image 908 show an exemplary plain PDMS substrate and an exemplary cell-imprinted PDMS substrate without culture of hiPSCs. Microscopic image 904 and microscopic image 906 show an exemplary plain PDMS substrate while culturing hiPSCs (after 3 days). Microscopic image 910 and microscopic image 912 show exemplary cell-imprinted PDMS substrates while culturing hiPSCs (after 3 days).

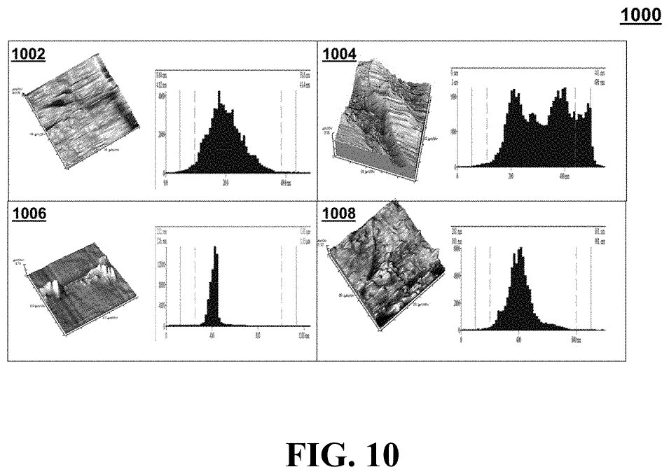

FIG. 10 illustrates AFM images 1000 of an exemplary plain PDMS substrate and an exemplary cell-imprinted PDMS substrate, consistent with one or more exemplary embodiments of the present disclosure. Regarding FIG. 10, AFM image 1002 illustrates topographical features of an exemplary plain PDMS substrate, AFM image 1004 illustrates topographical features of an exemplary cell-imprinted PDMS substrate, AFM image 1006 illustrates topographical features of an exemplary plain PDMS substrate during culture of hiPSCs, and AFM image 1008 illustrates topographical features of an exemplary cell-imprinted during culture of hiPSCs. Exemplary topographical features in AFM image 1004 and AFM image 1008 indicate exemplary areas on an exemplary cell-imprinted PDMS substrate that may have hfCMs pattern. Light microscopy, SEM, and AFM demonstrated the imprint of hfCMs topography on the surface of an exemplary cell-imprinted PDMS substrate. Meanwhile, microscopy analysis showed that hiPSCs were successfully embedded inside exemplary imprinted patterns of hfCMs on the surface of an exemplary PDMS substrate, during the culture of hfCMs.

### Example 5: Biochemical Surface Modification of Exemplary PDMS Substrates

In this example, exemplary PDMS substrates were coated/modified with exemplary biochemical agents. Two strategies were implemented to reduce the inherent hydrophobicity of PDMS: plasma treatment (PL treatment) and polydopamine coating (PD coating). For oxygen plasma treatment (plasma treatment), the cell-contacting surface (i.e., the surface to be in contact with cells) of exemplary topographically-modified PDMS substrates (i.e., cell-imprinted PDMS substrates) was exposed to plasma for about 90 seconds under low pressure and full power (40 kHz, 100 w). Subsequently, exemplary PL treated substrates were sterilized under UV light (for 30 minutes) and washed 3 times with deionized water.

To modify the cell-contacting surface of exemplary topographically-modified PDMS substrates (i.e., cell-imprinted substrates) with polydopamine, exemplary cell-imprinted substrates were immersed in an exemplary dopamine solution (0.01% w/v in Tris-HCl, pH 8.5) for about 8-24 hours. Exemplary PD-treated substrates were washed twice with deionized water and sterilized under UV light for about 30 seconds.

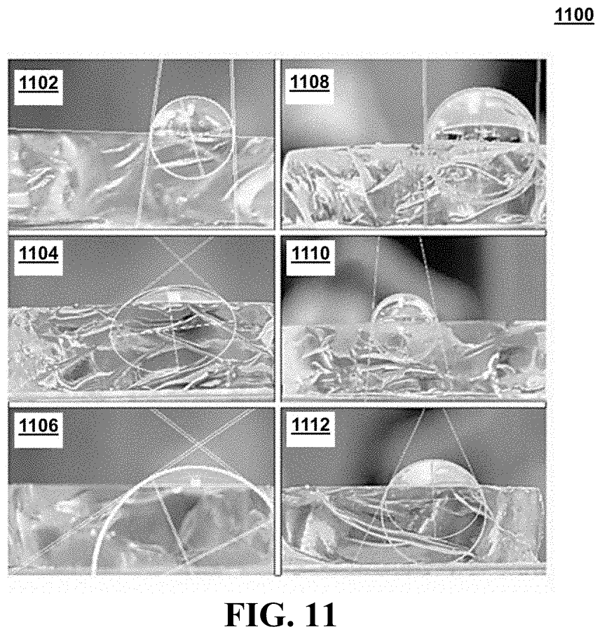

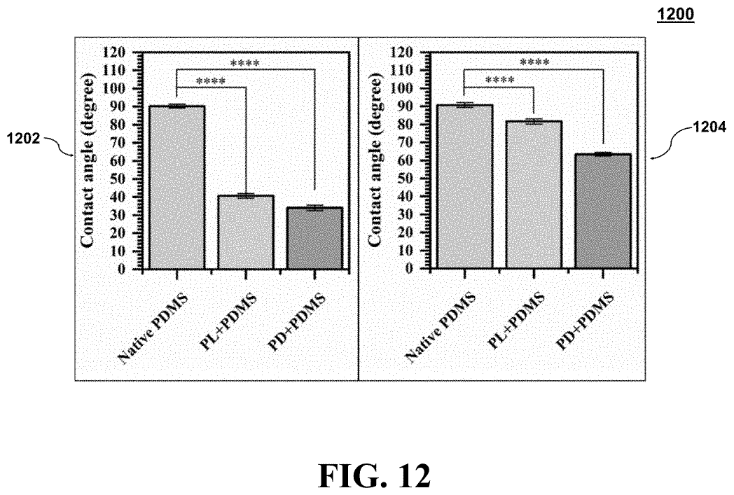

Surface hydrophobicity of exemplary PL-treated and PD-treated substrates was assessed by measuring water contact angle. Briefly, exemplary water droplets were formed on an exemplary modified surface of exemplary PL- and PD-treated substrates by slowly dropping 5 μL deionized water. The configuration of exemplary water droplets was captured using a high-resolution camera during 7 days. The contact angle of exemplary water droplets with respect to the modified surface of exemplary PL- and PD-treated substrates was measured using an image analysis software. FIG. 11 illustrates exemplary snapshots 1100 of an exemplary configuration of water droplets floating on a cell-contacting surface of an exemplary native/plain PDMS substrate, an exemplary PL-treated PDMS substrate, and an exemplary PD-coated PDMS substrate, consistent with one or more exemplary embodiments of the present disclosure. Regarding FIG. 11, snapshot 1102 shows an exemplary configuration of a water droplet on the surface of an exemplary plain PDMS substrate, snapshot 1104 shows an exemplary configuration of a water droplet on the modified surface of an exemplary PL-treated PDMS substrate, snapshot 1106 shows an exemplary configuration of a water droplet on the modified surface of an exemplary PD-coated PDMS substrate on day 0 (i.e., instantly after forming water droplets), snapshot 1108 shows an exemplary configuration of a water droplet on the surface of an exemplary plain PDMS substrate after 7 days, snapshot 1110 shows an exemplary configuration of a water droplet on the modified surface of an exemplary plasma-treated PDMS substrate after 7 days, and snapshot 1112 shows an exemplary configuration of a water droplet on the modified surface of an exemplary PD-coated PDMS substrate after 7 days. FIG. 12 illustrates charts 1200 of contact angle analysis of water droplets with respect to the cell-contacting surface of an exemplary plain/native PDMS substrate, an exemplary PL-treated PDMS substrate, and an exemplary PD-coated PDMS substrate immediately after forming water droplets (day 0) and after 7 days, consistent with one or more exemplary embodiments of the present disclosure. Regarding FIG. 12, chart 1202 shows and compares exemplary contact angles of water droplets on an exemplary plain/native PDMS substrate, an exemplary PL-treated PDMS substrate (PL+PDMS), and an exemplary PD-coated PDMS substrate on day 0. Chart 1204 shows and compares exemplary contact angles of water droplets on an exemplary plain/native PDMS substrate, an exemplary PL-treated PDMS substrate (PL+PDMS), and an exemplary PD-coated PDMS substrate after 7 days. ‘****’ shown in chart 1202 and chart 1204 may refer to P value<0.0001. As shown in FIG. 12, plasma treatment of PDMS substrate may reduce contact angle from 88.5±1.20° to 40.73±1.23° and polydopamine coating may decrease contact angle from 88.5±1.20° to 33.3±1.32° (P value<0.0001). After 7 days, hydrophobicity of an exemplary PD-coated substrate may be recovered from 33.3±1.32° to 63.43±1.32° that may be less than the hydrophobicity of an exemplary PL-treated PDMS substrate (from 40.73±1.23° to 81.63±1.23°, P value<0.0001). Charts 1200 demonstrates that an exemplary PD-coated PDMS substrate may be significantly more hydrophilic than an exemplary PL-treated PDMS substrate.

In an exemplary implementation, the modified surfaces of exemplary PL-treated and PD-coated PDMS substrates were further coated with a plurality of exemplary extra cellular matrix (ECM) proteins. Thereby, a plurality of exemplary PDMS substrates coated with ECM proteins were prepared using an exemplary untreated PDMS substrate (i.e., an exemplary PDMS substrate without plasma treatment and/or polydopamine coating). An exemplary untreated PDMS substrate were prepared with different coatings including: i) truncated Vitronectin (VTN-N), ii) Laminin-511 E8 fragment (LME8), and iii) Matrigel® (MG). MG refers to a solubilized basement membrane matrix secreted by Engelbreth-Holm-Swarm mouse sarcoma cells. ECM protein coating—including truncated VTN-N coating, LME8 coating, and MG coating—was also applied on an exemplary PL-treated PDMS substrate and an exemplary PD-coated PDMS substrate. Thus, considering exemplary control conditions, 12 exemplary substrates were prepared. Table 2 below sets forth exemplary PDMS substrates prepared with or without ECM protein coatings.

TABLE 2
Exemplary PDMS substrates with or without biochemical modifications
and extracellular matrix (ECM) protein coatings, consistent with
one or more exemplary embodiments of the present disclosure.

| Substrate | ECM coating |
|---|---|
| Untreated PDMS | No ECM coating |
| | Laminin E8 fragments |
| | Truncated vitronectin |
| | Matrigel ® |
| Plasma-treated PDMS | No ECM coating |
| | Laminin E8 fragments |
| | Truncated vitronectin |
| | Matrigel ® |
| Polydopamine-coated PDMS | No ECM coating |
| | Laminin E8 fragments |
| | Truncated vitronectin |
| | Matrigel ® |

### Example 6: Culturing hfCMs on the Surface of Biochemically-Modified PDMS Substrates

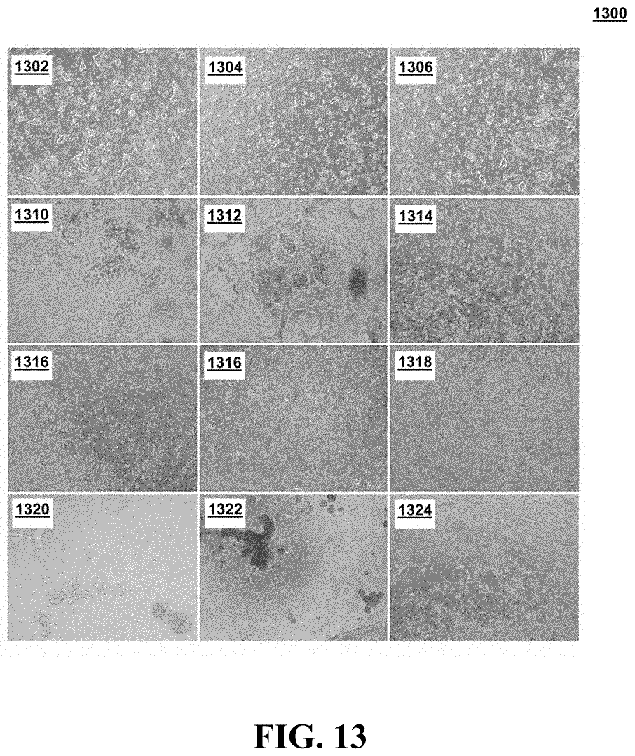

In this example, hiPSCs were cultured—using an exemplary method as set forth in ‘Example 2’—on the cell-contacting surface of exemplary substrates including TCPS (as control) and exemplary substrates set forth in Table 2. To examine the attachment/adhesion of hiPSCs to the cell-contacting surface of exemplary PDMS substrates, hiPSCs were seeded at a density of about 1×10⁵ cell/cm² on an exemplary TCPS and exemplary substrates set forth in Table 2. After culturing hiPSCs for 5 days, their adhesion and distribution were analyzed using an inverted microscope equipped with a digital camera. FIG. 13 illustrates light microscopy images 1300 of exemplary cultured hiPSCs on the cell-contacting surface of exemplary untreated and biochemically-modified PDMS substrates, consistent with one or more exemplary embodiments of the present disclosure. Referring to FIG. 13, microscopic image 1302 shows an exemplary untreated PDMS substrate (i.e., without plasma or polydopamine modifications) without ECM protein coating, microscopic image 1304 shows an exemplary PL-treated PDMS substrate without ECM protein coating, microscopic image 1306 shows an exemplary PD-coated PDMS substrate without ECM protein coating, microscopic image 1308 shows an exemplary untreated PDMS substrate (i.e., without plasma or polydopamine modifications) with MG coating, microscopic image 1310 shows an exemplary PL-treated PDMS substrate with truncated MG coating, microscopic image 1312 shows an exemplary PD-coated PDMS substrate with MG coating, microscopic image 1314 shows an exemplary untreated PDMS substrate (i.e., without plasma or polydopamine modifications) with VTN-N coating, microscopic image 1316 shows an exemplary PL-treated PDMS substrate with VTN-N coating, microscopic image 1318 shows an exemplary PD-coated PDMS substrate with VTN-N coating, microscopic image 1320 shows an exemplary untreated PDMS substrate (i.e., without plasma or polydopamine modifications) with LME8 coating, microscopic image 1322 shows an exemplary PL-treated PDMS substrate with MG coating, and microscopic image 1324 shows an exemplary PD-coated PDMS substrate with LME8 coating.

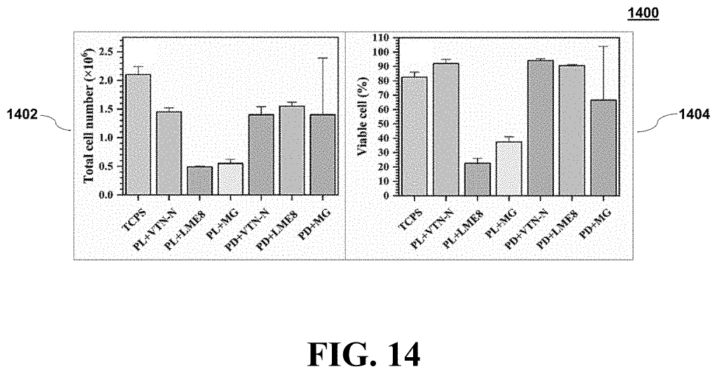

Proliferation of the cultured hiPSCs on the surface of exemplary PDMS substrates (on day 5) was also examined a cell counter device. FIG. 14 illustrates charts 1400 of proliferation and survival analysis of hiPSCs—based on total cell number and number of viable hiPSCs—after 5 days of culture on exemplary biochemically-modified PDMS substrates coated with exemplary ECM proteins, consistent with one or more exemplary embodiments of the present disclosure. Regarding FIG. 14, chart 1402 shows the total number of exemplary hiPSCs after a 5-day culture on exemplary substrates including an exemplary TCPS (as control), an exemplary PL-treated PDMS substrate with VTN-N coating, an exemplary PL-treated PDMS substrate with LME8 coating, an exemplary PL-treated PDMS substrate with MG coating, an exemplary PD-coated PDMS substrate coated with VTN-N, an exemplary PD-coated PDMS substrate with LME8 coating, and an exemplary PD-coated PDMS substrate with MG coating. Chart 1404 shows the number of exemplary viable hiPSCs after a 5-day culture on exemplary substrates including an exemplary TCPS (as control), an exemplary PL-treated PDMS substrate with VTN-N coating, an exemplary PL-treated PDMS substrate with LME8 coating, an exemplary PL-treated PDMS substrate with MG coating, an exemplary PD-coated PDMS substrate coated with VTN-N, an exemplary PD-coated PDMS substrate with LME8 coating, and an exemplary PD-coated PDMS substrate with MG coating. With further regards to FIG. 13, morphological observations demonstrated that all exemplary substrates without ECM coating (see microscopic images 1302, 1304, and 1306) were inefficient for hiPSCs adhesion. Meanwhile, exemplary hiPSCs did not show a uniform attachment to the cell-contacting surface of exemplary untreated PDMS substrates (i.e., without plasma or polydopamine modifications) with MG and LME8 coatings (see microscopic images 1308 and 1320) and exemplary attached cells were aggregated after 2-3 days. However, a uniform hiPSCs attachment was observed on the surface of an exemplary untreated PDMS substrate with VTN-N coating (see microscopic image 1314). On the other hand, cell adhesion was improved in both of exemplary PL-treated PDMS substrates with MG and LME8 coatings (see microscopic images 1310 and 1322)—compared to microscopic images 1302, 1304, 1306, and 1308). However, after 2-3 days, none of exemplary PL-treated PDMS substrates, with MG and LME8 coatings, maintained cell adhesion on their surface and exemplary hiPSCs started to detach from their surface. In comparison, an exemplary PL-treated PDMS substrate with VTN-N coating maintained cell adhesion for at least 5 days (see microscopic image 1318).

Exemplary PD-coated PDMS substrates with VTN-N coating and LME8 coating led to an efficient adhesion and maintenance of hiPSCs on their surface (see microscopic images 1318 and 1324). An exemplary PD-coated PDMS substrate with MG coating also led to a uniform and efficient attachment and expansion of exemplary hiPSCs, however—due to variations in the obtained results from exemplary proliferation and survival tests shown in FIG. 14—MG coating may not be an ideal coating for hiPSCs culture and differentiation. The obtained results suggested that polydopamine coating in combination with any of MG, VTN-N, and LME8 coatings may lead to efficient attachment and expansion of exemplary hiPSCs. Meanwhile, the obtained results demonstrated that amongst VTN-N, MG, and LME8, only VTN-N coating supported the attachment of hiPSCs to all exemplary substrates, including exemplary untreated, PL-treated, and PD-treated PDMS substrates, within 5 days. With further reference to FIGS. 13-14, based on the obtained results from morphological, proliferation, and survival studies, 3 exemplary PDMS substrates including an exemplary PL-treated PDMS substrate with VTN-N coating, an exemplary PD-coated PDMS substrate with VTN-N coating, and an exemplary PD-coated PDMS substrate with LME8 coating were selected for further experiments to obtain an exemplary ideal substrate for differentiation of hiPSCs to cardiomyocytes.

### Example 7: Evaluating Pluripotency Maintenance of hiPSCs on the Surface of Biochemically-Modified PDMS Substrates

In this example, pluripotency maintenance of hiPSCs on the surface of an exemplary PL-treated PDMS substrate with VTN-N coating, an exemplary PD-coated PDMS substrate with VTN-N coating, and an exemplary PD-coated PDMS substrate with LME8 coating was evaluated by flow cytometry. Assessment of pluripotency may be accomplished to ensure that biochemical modifications on PDMS do not lead to unwanted differentiation of hiPSCs.

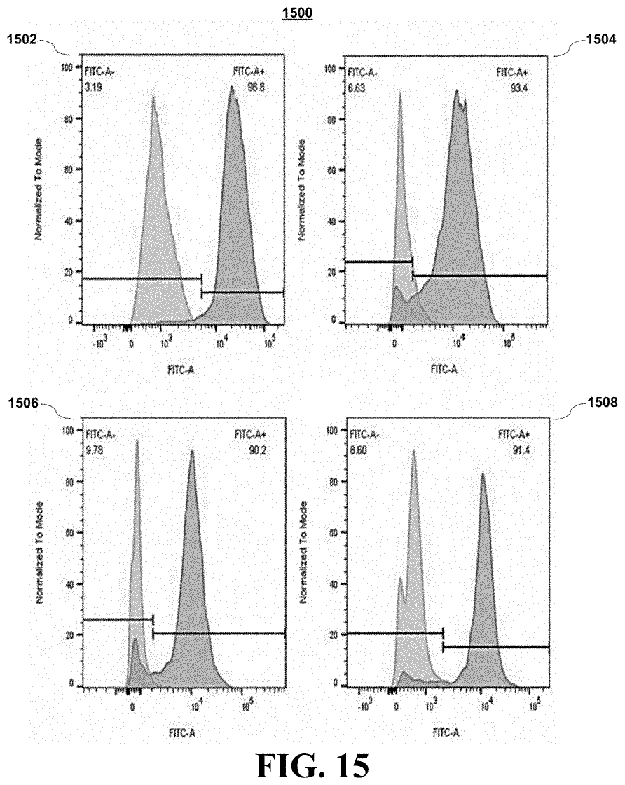

The presence of NANOG marker in stem cells (including hiPSCs) may be essential for maintaining pluripotency and self-renewal features. Thereby, in this example, expression of NANOG marker in exemplary hiPSCs was measured after a 5-day culture of exemplary hiPSCs on the modified surface of an exemplary PL-treated PDMS substrate with VTN-N coating, an exemplary PD-coated PDMS substrate with VTN-N coating, and an exemplary PD-coated PDMS substrate with LME8 coating. FIG. 15 illustrates graphs 1500 of flow cytometry analysis of exemplary NANOG-positive hiPSCs on fourth day of culture to assess pluripotency maintenance of exemplary hiPSCs on the modified surface of an exemplary PL-treated PDMS substrate with VTN-N coating, an exemplary PD-coated PDMS substrate with VTN-N coating, an exemplary PD-coated PDMS substrate with LME8 coating, and an exemplary TCPS (as control), consistent with one or more exemplary embodiments of the present disclosure. In further detail with respect to graphs 1500, graph 1502 shows flow cytometry analysis of exemplary NANOG-positive hiPSCs cultured on the modified surface of an exemplary PL-treated PDMS substrate with VTN-N coating, graph 1504 shows flow cytometry analysis of exemplary NANOG-positive hiPSCs cultured on the modified surface of an exemplary PD-coated PDMS substrate with VTN-N coating, graph 1506 shows flow cytometry analysis of exemplary NANOG-positive hiPSCs cultured on the modified surface of an exemplary PD-coated PDMS substrate with LME8 coating, and graph 1508 shows flow cytometry analysis of exemplary NANOG-positive hiPSCs cultured on the modified surface of an exemplary TCPS (as control).

Graphs 1500 demonstrated that the amount of NANOG expressed by exemplary hiPSCs cultured on the modified surface of an exemplary PL-treated PDMS substrate with VTN-N coating, an exemplary PD-coated PDMS substrate with VTN-N coating, and an exemplary PD-coated PDMS substrate with LME8 coating was similar to the amount of NANOG expressed by exemplary hiPSCs cultured on an exemplary TCPS (as an exemplary control condition). Furthermore, graphs 1500 demonstrated that culturing exemplary hiPSCs on an exemplary PL-treated PDMS substrate with VTN-N coating, an exemplary PD-coated PDMS substrate with VTN-N coating, and an exemplary PD-coated PDMS substrate with LME8 substrates may result in an efficient attachment, proliferation, and pluripotency maintenance of exemplary hiPSCs.

### Example 8: Differentiation of hiPSCs to Cardiomyocytes on Biochemically-Modified PDMS Substrates

In this example, hiPSCs were differentiated to cardiomyocytes on the modified surface of an exemplary PL-treated PDMS substrate with VTN-N coating, an exemplary PD-coated PDMS substrate with VTN-N coating, and an exemplary PD-coated PDMS substrate with LME8 coating using an exemplary method described in one or more exemplary embodiments.

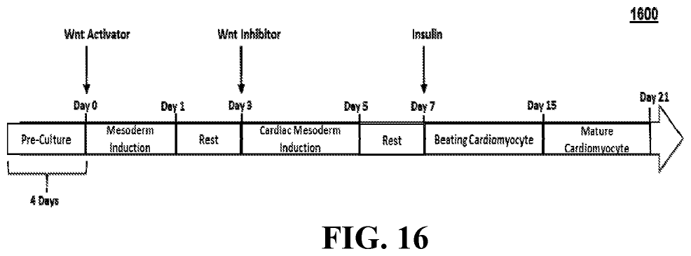

Briefly, about 1×10⁵ hiPSCs were seeded on an exemplary LME8-coated TCPS (as an exemplary control), an exemplary untreated PDMS substrate as an exemplary control (i.e., an exemplary PDMS substrate without plasma treatment, polydopamine coating, and ECM protein coating), and exemplary biochemically-modified PDMS substrates including an exemplary PL-treated PDMS substrate with VTN-N coating, an exemplary PD-coated PDMS substrate with VTN-N coating, and an exemplary PD-coated PDMS substrate with LME8 coating. Following hiPSCs seeding, exemplary hiPSCs were cultured during a 4-day preculture step before entering into an exemplary differentiation process. On day (1) of the 4-days preculture step, exemplary hiPSCs may be cultured using an exemplary Essential 8™ medium comprising 10 μM Y-27632 (a selective inhibitor of Rho-associated coiled-coil containing protein kinase (ROCK)). After 24 hours, E8 medium was removed and substituted with a fresh E8 medium (Y-27632 may not be added again after the first day of preculture). E8 medium may be changed every day until day (4) of the 4-days preculture. After 4 days of preculturing, when exemplary hiPSCs reached a confluency of about 90-100%, differentiation process was started by adding an exemplary differentiation medium comprising B27™ supplemented RPMI (Roswell Park Memorial Institute Medium) and 12 μM CHIR99021 to induce differentiation of exemplary hiPSCs to mesoderm lineage. After 24 hours, an exemplary differentiation medium was removed and replaced with an exemplary CHIR99021-free medium. On day (3) of differentiation, WNT inhibitor (XAV939 with a final concentration of about 2 μM) was added to an exemplary CHIR99021-free medium and the entire supernatant (i.e., CHIR99021-free medium plus WNT inhibitor) was removed after 48 hours. From day (7) until the end of differentiation process, exemplary hiPSCs were incubated with an exemplary CHIR99021-free medium comprising about 1 μg insulin. FIG. 16 illustrates timeline chart 1600 of an exemplary procedure of cardiomyocyte differentiation and maturation, consistent with one or more exemplary embodiments of the present disclosure. As shown in time line chart 1600, it was expected to visualize cell beating on days 7 to 14.

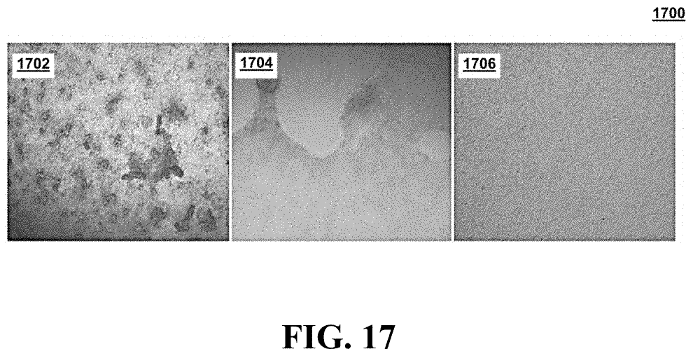

FIG. 17 illustrates light microscopy images 1700 of exemplary hiPSCs after mesoderm induction on the modified surface of an exemplary PL-treated PDMS substrate with VTN-N coating, an exemplary PD-coated PDMS substrate with VTN-N coating, and an exemplary PD-coated PDMS substrate with LME8 coating, consistent with one or more exemplary embodiments of the present disclosure. Referring to FIG. 17, microscopic image 1702 shows exemplary hiPSCs after mesoderm induction on the modified surface of PL-treated PDMS substrate with VTN-N coating, microscopic image 1704 shows exemplary hiPSCs after mesoderm induction on the modified surface of an exemplary PD-coated PDMS substrate with VTN-N coating, and microscopic image 1706 shows exemplary hiPSCs after mesoderm induction on the modified surface of an exemplary PD-coated PDMS substrate with LME8 coating. Light microscopy images 1700 demonstrated that after treating exemplary hiPSCs with CHIR99021, exemplary hiPSCs were detached from exemplary PL-treated and PD-coated PDMS substrates with VTN-N coating. However, an exemplary PD-coated PDMS substrate with LME8 coating led to a stable attachment of exemplary hiPSCs during mesoderm induction and the entire process of differentiation (at least 25 days).

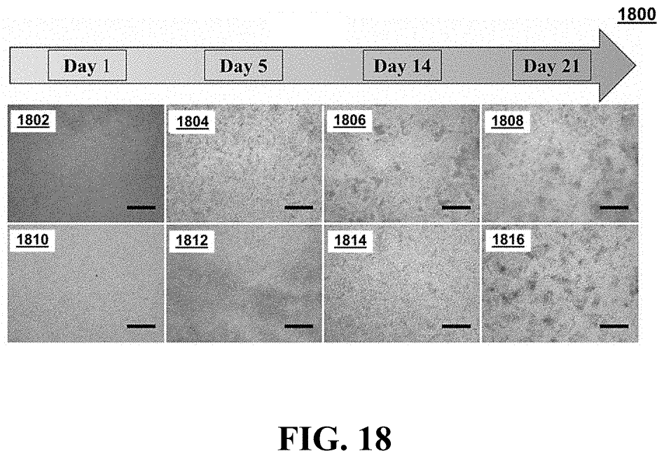

FIG. 18 illustrates light microscopy images 1800 of exemplary hiPSCs during their differentiation to cardiomyocytes (on day (1), day (5), day (14), and day (21) of differentiation) on the modified surface of an exemplary PD-coated PDMS substrate with LME8 coating and an exemplary LME8-coated TCPS (as an exemplary control), consistent with one or more exemplary embodiments of the present disclosure. Referring to FIG. 18, microscopic image 1802 and microscopic image 1810, respectively, demonstrate the induction of exemplary hiPSCs to mesoderm on the modified surface of an exemplary PD-coated PDMS substrate with LME8 coating and an exemplary LME8-coated TCPSon day (1). Microscopic image 1804 and microscopic image 1812, respectively, demonstrate mesoderm differentiation to cardiac mesoderm on the modified surface of an exemplary PD-coated PDMS substrate with LME8 coating and an exemplary LME8-coated TCPS on day (5). Microscopic image 1806 and microscopic image 1814, respectively, demonstrate cardiac mesoderm differentiation to immature cardiomyocytes on the modified surface of an exemplary PD-coated PDMS substrate with LME8 coating and an exemplary LME8-coated TCPS on day (14). Microscopic image 1808 and microscopic image 1816, respectively, demonstrate the differentiation of immature cardiomyocytes to mature and beating cardiomyocytes on the modified surface of an exemplary PD-coated PDMS substrate with LME8 coating and an exemplary LME8-coated TCPS on day (21). Light microscopy images 1700 and light microscopy images 1800 demonstrated that an exemplary PD-coated PDMS substrate with LME8 coating, in comparison to an exemplary PL-treated PDMS substrate with VTN-N coating and an exemplary PD-coated PDMS substrate with VTN-N coating, was ideal for maintaining exemplary hiPSCs stably attached to the cell-contacting surface both during preculture phase and during differentiation process. Thus, an exemplary PD-coated PDMS substrate with LME8 coating may be an ideal substrate that may provide for integration of an exemplary process of hiPSCs proliferation/expansion with an exemplary process of hiPSCs differentiation to cardiomyocytes.

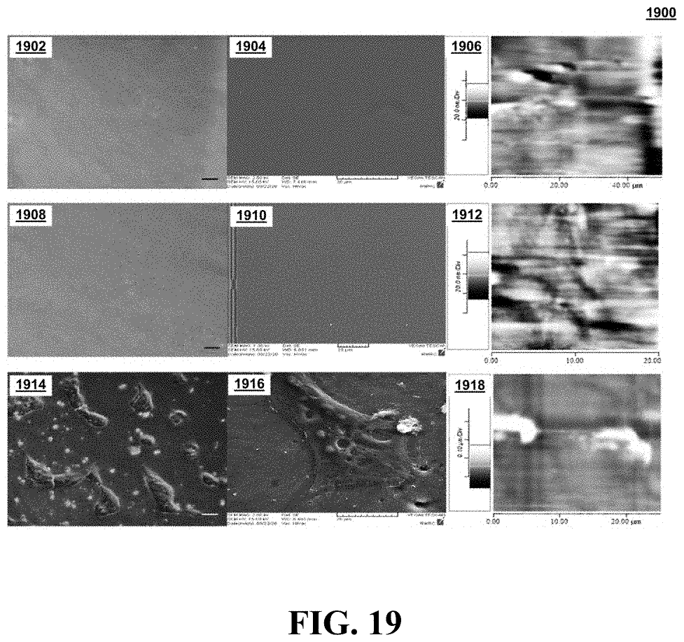

Topographical features of an exemplary PD-coated PDMS substrate with LME8 coating during the proliferation/expansion and differentiation of exemplary hiPSCs was investigated by light microscopy, AFM, and SEM. FIG. 19 illustrates images 1900 of surface characterization of an exemplary PD-coated PDMS substrate with LME8 coating compared to an exemplary PDMS substrate without biochemical and ECM protein modifications by light microscopy, SEM, and AFM, consistent with one or more exemplary embodiments of the present disclosure. Microscopic image 1902 shows the surface of an exemplary PDMS substrate without biochemical and ECM protein modifications (×20 magnifications), SEM image 1904 shows the surface of an exemplary PDMS substrate without biochemical and ECM protein modifications, AFM image 1906 shows the surface of an exemplary PDMS substrate without biochemical and ECM protein modifications, microscopic image 1908 shows the modified surface of an exemplary PD-coated PDMS substrate with LME8 coating (×20 magnification), SEM image 1910 shows the modified surface of an exemplary PD-coated PDMS substrate with LME8 coating, AFM image 1912 shows the modified surface of an exemplary PD-coated PDMS substrate with LME8 coating, microscopic image 1914 shows the modified surface of an exemplary PD-coated PDMS substrate with LME8 coating during hiPSCs expansion (×20 magnification), SEM image 1916 shows the modified surface of an exemplary PD-coated PDMS substrate with LME8 coating during hiPSCs expansion, and AFM image 1918 shows the modified surface of an exemplary PD-coated PDMS substrate with LME8 coating during hiPSCs expansion.

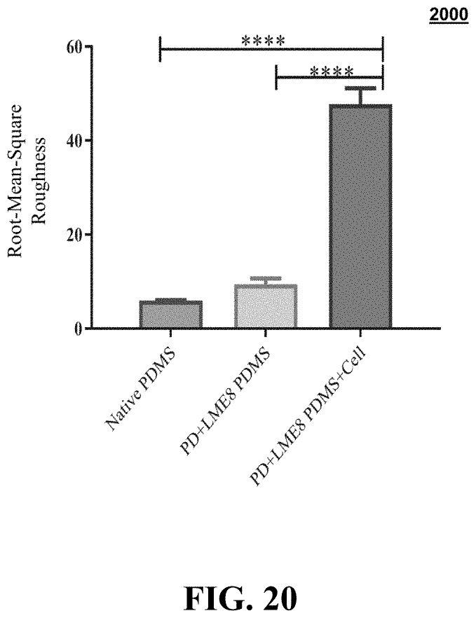

FIG. 20 illustrates bar graph 2000 of root-mean-square (RMS) roughness analysis of an exemplary PD-coated PDMS substrate with LME8 coating without exemplary hiPSCs, an exemplary PDMS substrate without biochemical and ECM protein modification, and an exemplary PD-coated PDMS substrate with LME8 coating during expansion of exemplary hiPSCs, consistent with one or more exemplary embodiments of the present disclosure. ‘*’ and ‘**’ in FIG. 20 may refer to P-value<0.05 and P-value<0.01, respectively. Bar graph 2000 demonstrates that the modified surface of an exemplary PD-coated PDMS substrate with LME8 coating may have a significantly higher roughness compared to the surface of an exemplary PDMS substrate without biochemical and ECM protein modification. Meanwhile, the modified surface of an exemplary PD-coated PDMS substrate with LME8 coating during expansion of hiPSCs may have a significantly higher roughness compared to the modified surface of an exemplary PD-coated PDMS substrate with LME8 coating without exemplary hiPSCs.

The obtained results in this example demonstrated that hiPSCs morphology and adhesion (both during proliferation and differentiation) on the modified surface of an exemplary PD-coated PDMS substrate with LME8 coating was capable of maintaining exemplary hiPSCs stably attached to the cell-contacting surface during the entire process of proliferation and differentiation—similar to an exemplary control condition (i.e., the LME8-coated TCPS). Thus, using an exemplary method described in one or more exemplary embodiments, both procedures of hiPSCs culture and differentiation may be accomplished on a single substrate.

### Example 9: Analysis of Gene Expression During Differentiation of hiPSCs to Cardiomyocytes

In this example, expression level of cardiomyocytes' differentiation genes was measured (on day (1), day (8), and day (21) of differentiation) by quantitative polymerase chain reaction (qPCR) to validate an exemplary PD-coated PDMS substrate with LME8 coating as an ideal substrate for differentiation of exemplary hiPSCs to cardiomyocytes in an integrated process. Analyzed gene markers include NANOG (marker of hiPSCs pluripotency), Homeobox protein NKX2-5 gene (NKX2-5) and neural crest-derived transcript-1 gene (HAND1) (markers of cardiac mesoderm formation), Brachyury gene (T) (marker of mesoderm formation), cardiac troponin T (TNNT2) (marker of cardiac differentiation), and myosin heavy chain 7 gene (MYH7), myosin heavy chain 7 (MYH6), and cardiac troponin I gene (TNNI3) (markers of cardiac maturation). Glyceraldehyde 3-phosphate dehydrogenase (GAPDH) was used as internal control.

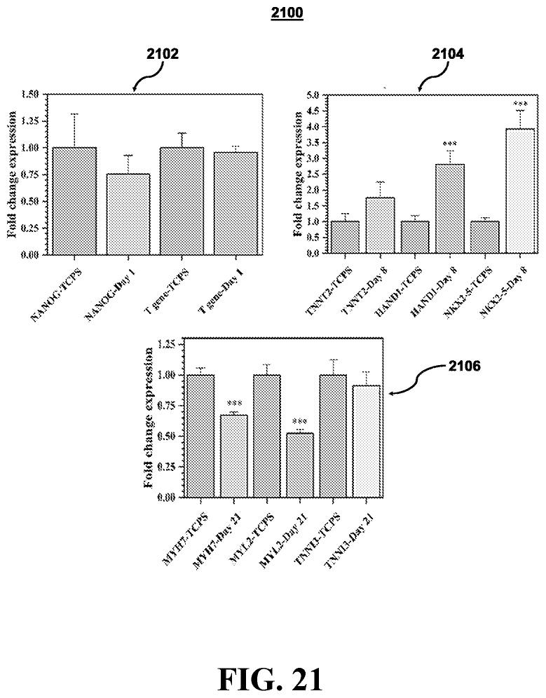

FIG. 21 illustrates charts 2100 of expression level analysis of exemplary differentiation genes during differentiation of exemplary hiPSCs to cardiomyocytes on the modified surface of an exemplary PD-coated PDMS substrate with LME8 coating, compared to the expression level of exemplary differentiation genes during differentiation of exemplary hiPSCs to cardiomyocytes on the modified surface of an exemplary LME8-coated TCPS (as an exemplary control), consistent with one or more exemplary embodiments of the present disclosure. In further detail with respect to charts 2100, chart 2102 shows the expression level of NANOG and T gene after CHIR99021 treatment—on day (1)—on the modified surface of an exemplary PD-coated PDMS substrate with LME8 coating and an exemplary LME8-coated TCPS. Chart 2104 shows the expression level of NKX2-5, HAND1, and TNNT2 (after day (8) of differentiation) on the modified surface of an exemplary PD-coated PDMS substrate with LME8 coating and an exemplary LME8-coated TCPS. Chart 2106 shows the expression level of MYH6, TNNT2, and MYH7 (on day (21) of differentiation) on the modified surface of an exemplary PD-coated PDMS substrate with LME8 coating and an exemplary LME8-coated TCPS. ‘***’ shown in charts 2100 may refer to P-value<0.001.

Comparing exemplary levels of expressed genes—during differentiation—on the modified surface of an exemplary PD-coated PDMS substrate with LME8 coating and an exemplary control condition (i.e., an exemplary LME8-coated TCPS), no significant variations were observed between an exemplary PD-coated PDMS substrate with LME8 coating and an exemplary control condition, in the initiation stage of mesoderm (on day (1), chart 2102) and in the cardiac maturation stage (on day (21), chart 2106). In particular, gene expression alterations in the produced cardiomyocytes on an exemplary PD-coated PDMS substrate with LME8 coating were less than 1-fold comparing to the produced cardiomyocytes over an exemplary LME8-coated TCPS—in the initiation stage of mesoderm (on day (1)) and in the cardiac maturation stage (on day (21)).

Although all exemplary genes (examined in this example) were expressed on both of an exemplary PD-coated PDMS substrate with LME8 coating and an exemplary control condition, in the progenitor stage (on day (8)), the expression level of exemplary early cardiac marker genes (HAND1 and NKX2-5) and cardiomyocyte marker (TNNT2) in the produced cardiomyocytes on an exemplary PD-coated PDMS substrate with LME8 coating was significantly higher than an exemplary control condition (2.82 folds for HAND1, 3.93 folds for NKX2-5, and 1.78 folds for TNNT2). Furthermore, charts 2100 demonstrated that the level of exemplary genes expressed during differentiation of exemplary hiPSCs to cardiomyocytes on an exemplary PD-coated PDMS substrate with LME8 coating was similar to an exemplary control condition.

### Example 10: Protein Expression Analysis During Differentiation of hiPSCs to Cardiomyocytes

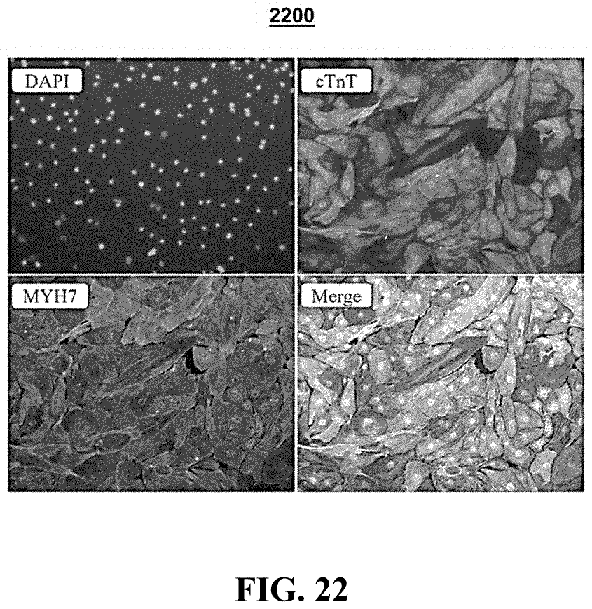

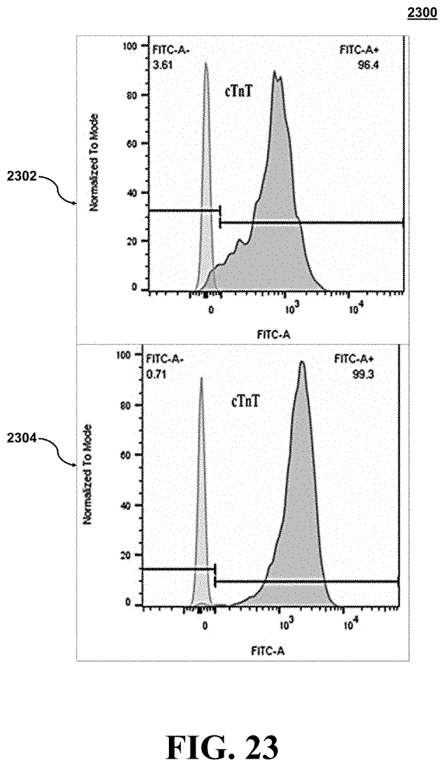

In this example, expression level of exemplary pluripotency markers (NANOG and OCT-3/4), an exemplary marker of hiPSCs-derived cardiomyocytes (cardiac troponin T protein (cTnT)), and an exemplary cardiac maturation marker (cardiac myosin heavy chain protein beta (MYH7)) was evaluated in exemplary produced cardiomyocytes by immunostaining and flow cytometry analysis. Expression of cTnT was measured by flow cytometry on day (14) and day (50) from the initiation of differentiation phase. Meanwhile, expression of hiPSCs-derived cardiomyocytes was evaluated by detecting cTnT and MYH7 by immunostaining/ICC after 50 days of differentiation. FIG. 22 illustrates microscopic images 2200 of immunostaining assay of exemplary cardiac markers, MYH7, and cTnT on day (50) of cardiac differentiation, consistent with one or more exemplary embodiments of the present disclosure. FIG. 23 illustrates graphs 2300 of flow cytometry analysis of hiPSCs-derived cardiomyocytes to evaluate cTnT expression, consistent with one or more exemplary embodiments of the present disclosure. In further detail with respect to graphs 2300, graph 2302 demonstrates flow cytometry analysis of cTnT expression on day (14) of differentiation, and graph 2304 demonstrates flow cytometry analysis of cTnT expression on day (50) of differentiation. As shown in graph 2306, in cardiac maturation stage (on day 21), the level of exemplary cardiomyocyte-specific markers including TNNT2, MYH7, MYL2, and TNNI3 genes expressed in exemplary hiPSCs-derived cardiomyocytes harvested from exemplary PDMS substrates set forth in Table 2 was not significantly different from an exemplary control condition (an exemplary LME8-coated TCPS).

### Example 11: Evaluating the Effect of Physicochemical Properties of Substrate on Production of hiPSCs-Derived Cardiomyocytes in an Integrated Process

In this example, after identifying an ideal biochemical modification for an exemplary PDMS substrate, production of exemplary hiPSCs-derived cardiomyocytes was evaluated on exemplary substrates set forth in Table 1, all of which were prepare with polydopamine and LME8 coatings. Exemplary examined substrates in this example include an exemplary cell-imprinted/topographically-modified PDMS substrate with 667 kPa (1:10) stiffness, an exemplary plain PDMS substrate (i.e., without topographical modification) with 667 kPa (1:10) stiffness, an exemplary cell-imprinted PDMS substrate with 106 kPa (1:30) stiffness, and an exemplary plain PDMS substrate (i.e., without topographical modification) with 106 kPa (1:30) stiffness, all of which were coated with polydopamine and LME8. An exemplary LME8-coated TCPS was used as an exemplary control condition.

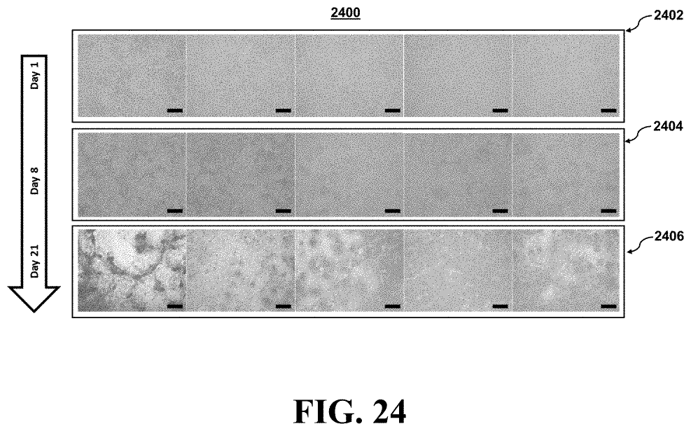

FIG. 24 illustrates light microscopy images 2400 captured from the modified surface of an exemplary cell-imprinted PDMS substrate with 667 kPa (1:10) and 106 kPa (1:30) stiffness and the surface of an exemplary plain PDMS substrate (i.e., without topographical modification) with 667 kPa (1:10) and 106 kPa (1:30) stiffness (all of them coated with polydopamine and LME8) during differentiation of exemplary hiPSCs to cardiomyocytes (on days (1), (8), and (21)), consistent with one or more exemplary embodiments of the present disclosure. Referring to light microscopy images 2400, microscopic images 2402 show the morphology of mesoderm after mesoderm induction (day (1) of differentiation) over an exemplary cell-imprinted PDMS substrate with 667 kPa (1:10) stiffness, an exemplary plain PDMS substrate with 667 kPa (1:10) stiffness, an exemplary cell-imprinted PDMS substrate with 106 kPa (1:30) stiffness, and an exemplary plain PDMS substrate with 106 kPa (1:30) stiffness (all of them coated with polydopamine and LME8). Microscopic images 2404 show the morphology of exemplary progenitor cardiomyocytes on day (8) of differentiation over an exemplary cell-imprinted PDMS substrate with 667 kPa (1:10) stiffness, an exemplary plain PDMS substrate with 667 kPa (1:10) stiffness, an exemplary cell-imprinted PDMS substrate with 106 kPa (1:30) stiffness, and an exemplary plain PDMS substrate with 106 kPa (1:30) stiffness (all of them coated with polydopamine and LME8). Microscopic images 2406 show the morphology of exemplary mature cardiomyocytes on day (21) of differentiation over an exemplary cell-imprinted PDMS substrate with 667 kPa (1:10) stiffness, an exemplary plain PDMS substrate with 667 kPa (1:10) stiffness, an exemplary cell-imprinted PDMS substrate with 106 kPa (1:30) stiffness, and an exemplary plain PDMS substrate with 106 kPa (1:30) stiffness (all of them coated with polydopamine and LME8).

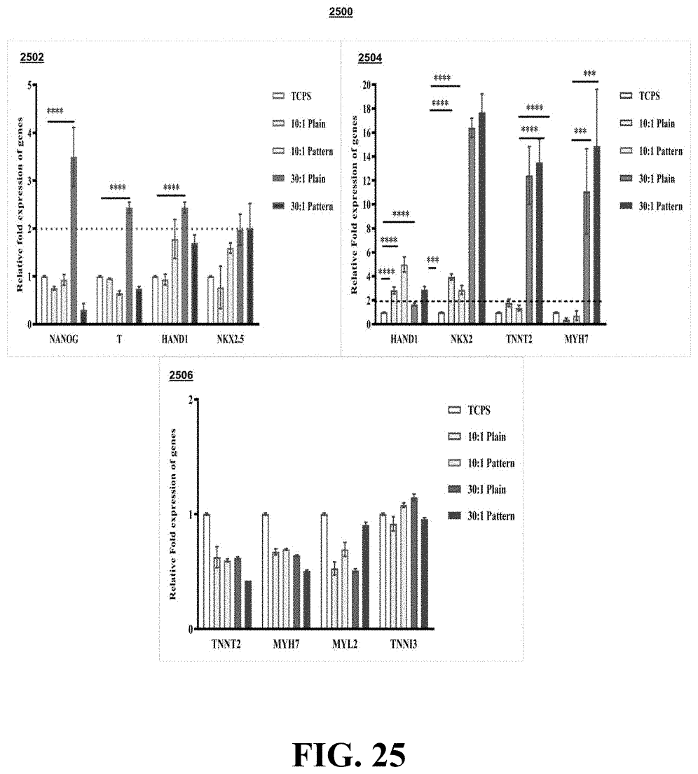

FIG. 25 illustrates charts 2500 of expression level analysis of exemplary differentiation genes during differentiation of exemplary hiPSCs to cardiomyocytes over an exemplary cell-imprinted PDMS substrate with 667 kPa (1:10) and 106 kPa (1:30) stiffness and an exemplary plain PDMS substrate with 667 kPa (1:10) and 106 kPa (1:30) stiffness (all of them coated with polydopamine and LME8), consistent with one or more exemplary embodiments of the present disclosure. In further detail with respect to charts 2500, chart 2502 shows the expression level of NANOG gene, T gene, HAND1 gene, and NKX2.5 gene after mesoderm induction on day (1). Chart 2504 shows the expression level of HAND1, NKX2-5, ACTN2, TNNT2, and MYH7 genes after day (8) of differentiation. Chart 2506 shows the expression level of TNNT2, MYH7, MYL2, and TNNI3 genes on day (21) of differentiation. ‘***’ shown in charts 2500 may refer to P-value<0.001.

With further regards to FIG. 25, on day 1 (mesoderm induction) pluripotency marker gene (NANOG), mesodermal marker gene (T gene), and cardiac mesoderm marker (HAND1) significantly increased in the harvested cardiomyocytes from the surface of an exemplary plain PDMS substrate with 106 kPa (1:30) stiffness compared to other exemplary substrates set forth in FIG. 25. In the stage of cardiac progenitor production (on day 8)— although the expression level of all exemplary differentiation genes were increased in both of exemplary plain and cell-imprinted PDMS substrates with 1:10 and 1:30 stiffness compared to an exemplary LME8-coated TCPS—the cardiac progenitors harvested from the surface of exemplary substrates with 1:30 stiffness demonstrated a significantly higher level of gene expression (i.e., HAND1, NKX2-5, ACTN2, TNNT2, and MYH7 genes). Early increase of MYH7 on day (8) may lead to faster generation of mature cardiomyocytes. As demonstrated in charts 2500, an exemplary cell-imprinted PDMS substrate with 1:30 stiffness (being coated with polydopamine and LME8) significantly enhanced expression of NKX2.5, TNNT2, and MYH7 compared to an exemplary plain PDMS substrate with 1:10 stiffness (HAND1, NKX2-5, ACTN2, TNNT2, and MYH7 genes). The obtained results (as described in Examples 6-11) demonstrated that imprinting micro/nano structure of human cardiomyocytes, preparing a PDMS substrate with a lower stiffness as set forth in one or more exemplary embodiments, and applying an exemplary biochemical coating as described in one or more exemplary embodiments may contribute to production of mature cardiomyocytes in an integrated process without risk of cells detachment from the cell-contacting surface of an exemplary PDMS substrate and/or cells aggregation during the expansion and differentiation of exemplary hiPSCs.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. An element proceeded by “a” or “an” does not, without further constraints, preclude the existence of additional identical elements in the process, method, article, or apparatus that comprises the element.

Unless otherwise stated, all measurements, values, ratings, positions, magnitudes, sizes, and other specifications that are set forth in this specification, are approximate, not exact. They are intended to have a reasonable range that is consistent with the functions to which they relate and with what is customary in the art to which they pertain.

It will be understood that the terms and expressions used herein have the ordinary meaning as is accorded to such terms and expressions with respect to their corresponding respective areas of inquiry and study, except where specific meanings have otherwise been set forth herein. Relational terms such as “first” and “second” and the like may be used solely to distinguish one entity or action from another without necessarily requiring or implying any actual such relationship or order between such entities or actions.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it may be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A method for generating a population of cardiomyocytes from induced pluripotent stem cells (iPSCs) in an integrated process, the method comprising:
seeding the iPSCs on a modified surface of a modified cell culture substrate, the modified cell culture substrate comprising:
a patterned polydimethylsiloxane (PDMS) substrate with a stiffness of 667 kPa, the patterned PDMS substrate comprising an imprinted pattern of human fetus cardiomyocytes on a cell-contacting surface of the patterned PDMS substrate;
a first coating comprising a plurality of polydopamine molecules chemically attached to the cell-contacting surface of the patterned PDMS substrate, wherein the plurality of polydopamine molecules have a coating concentration between 47 μg/cm² and 53 μg/cm²; and
a second coating covering a top surface of the first coating, the second coating comprising a plurality of Laminin 511 E8 Fragment (LME8) molecules chemically attached to the first coating, wherein the plurality of LME8 molecules have a coating concentration between 0.4 μg/cm² and 0.6 μg/cm²;
culturing the seeded iPSCs on the modified surface of the modified cell culture substrate in an animal component-free culture medium until reaching a confluency of at least 80%; and
differentiating the cultured iPSCs to the population of cardiomyocytes on the modified surface of the modified cell culture substrate comprising:
inducing differentiation of the cultured iPSCs to a population of mesoderm cells by culturing the cultured iPSCs in a first growth medium comprising a Wnt activator;
inducing differentiation of the population of mesoderm cells to a population of cardiac mesoderm cells by culturing the population of mesoderm cells in a second growth medium comprising a Wnt inhibitor; and
inducing differentiation of the population of cardiac mesoderm cells to the population of cardiomyocytes by culturing the population of cardiac mesoderm cells in a third growth medium comprising Insulin.

2. The method of claim 1, wherein the animal component-free culture medium comprises a Dulbecco's Modified Eagle Medium F-12 comprising at least one of L-ascorbic acid-2-phosphate magnesium, sodium selenium, fibroblast growth factor 2 (FGF2), Insulin, NaHCO₃, transferrin, and transforming growth factor beta 1 (TGFβ1).

3. The method of claim 1, wherein the animal component-free culture medium comprises an inhibitor of Rho-associated, coiled-coil containing protein kinase (ROCK) with a concentration between 8 μM and 12 μM.

4. The method of claim 1, wherein inducing differentiation of the cultured iPSCs to the population of mesoderm cells by culturing the cultured iPSCs in the first growth medium comprising the Wnt activator comprises inducing differentiation of the cultured iPSCs to the population of mesoderm cells by culturing the cultured iPSCs in Roswell Park Memorial Institute Medium (RPMI) comprising CHIR99021 with a concentration between 10 μM and 14 μM for a time duration between 18 and 30 hours.

5. The method of claim 1, wherein inducing differentiation of the population of mesoderm cells to the population of cardiac mesoderm cells by culturing the population of mesoderm cells in the second growth medium comprising the Wnt inhibitor comprises inducing differentiation of the population of mesoderm cells to the population of cardiac mesoderm cells by culturing the population of mesoderm cells in RPMI comprising XAV939 with a concentration between 1.5 μM and 2.5 μM for a time duration between 48 and 100 hours.

6. The method of claim 1, wherein inducing differentiation of the population of cardiac mesoderm cells to the population of cardiomyocytes by culturing the population of cardiac mesoderm cells in the third growth medium comprising Insulin comprises inducing differentiation of the population of cardiac mesoderm cells in RPMI comprising Insulin for a time duration between 5 and 15 days.
